(19) United States
(12) Patent Application Publication
(10) Pub. No.: US 2004/0224876 A1
(43) Pub. Date: Nov. 11, 2004

(54) Combination therapy for the treatment of immunoinflammatory disorders

(76) Inventors: Edward Roydon Jost-Price, West Roxbury, MA (US); Bradley B. Brasher, Natick, MA (US); Todd W. Chappell, Boston, MA (US); Palaniyandi Manivasakam, West Roxbury, MA (US); Noah Sachs, Boston, MA (US); Brendan Smith, Boston, MA (US); Benjamin A. Auspitz, Cambridge, MA (US)

Correspondence Address: CLARK & ELBING LLP, 101 FEDERAL STREET, BOSTON, MA, 02110, US

(21) Appl. No.: 10/777,518

(22) Filed: Feb. 12, 2004

Related U.S. Application Data
(60) Provisional application No. 60/447,366, filed on Feb. 14, 2003.
(60) Provisional application No. 60/447,412, filed on Feb. 14, 2003.
(60) Provisional application No. 60/447,415, filed on Feb. 14, 2003.
(60) Provisional application No. 60/447,553, filed on Feb. 14, 2003.
(60) Provisional application No. 60/447,648, filed on Feb. 14, 2003.
(60) Provisional application No. 60/464,753, filed on Apr. 23, 2003.
(60) Provisional application No. 60/503,026, filed on Sep. 15, 2003.

(51) Int. Cl. A61K 38/13

(52) U.S. Cl. 514/11

(57) ABSTRACT

The invention features a method for treating a patient diagnosed with, or at risk of developing, an immunoinflammatory disorder by administering a non-steroidal immunophilin-dependent immunosuppressant (NsIDI) and an NsIDI enhancer (NsIDIE) or analog or metabolite thereof to the patient. The invention also features a pharmaceutical composition containing an NsIDI and NsIDIE or analog or metabolite thereof for the treatment or prevention of an immunoinflammatory disorder.

### CROSS-REFERENCE TO RELATED APPLICATIONS

This application claims the benefit of U.S. Provisional Application Nos. 60/447,366, 60/447,412, 60/447,415, 60/447,553, and 60/447,648, each filed Feb. 14, 2003, 60/464,753, filed Apr. 23, 2003, and 60/503,026, filed on Sep. 15, 2003, each of which is hereby incorporated by reference.

### BACKGROUND OF THE INVENTION

The invention relates to the treatment of immunoinflammatory disorders.

Immunoinflammatory disorders are characterized by the inappropriate activation of the body's immune defenses. Rather than targeting infectious invaders, the immune response targets and damages the body's own tissues or transplanted tissues. The tissue targeted by the immune system varies with the disorder. For example, in multiple sclerosis, the immune response is directed against the neuronal tissue, while in Crohn's disease the digestive tract is targeted. Immunoinflammatory disorders affect millions of individuals and include conditions such as asthma, allergic intraocular inflammatory diseases, arthritis, atopic dermatitis, atopic eczema, diabetes, hemolytic anaemia, inflammatory dermatoses, inflammatory bowel or gastrointestinal disorders (e.g., Crohn's disease and ulcerative colitis), multiple sclerosis, myasthenia gravis, pruritis/inflammation, psoriasis, rheumatoid arthritis, cirrhosis, and systemic lupus erythematosus.

Current treatment regimens for immunoinflammatory disorders typically rely on immunosuppressive agents. The effectiveness of these agents can vary and their use is often accompanied by adverse side effects. Thus, improved therapeutic agents and methods for the treatment of immunoinflammatory disorders are needed.

### SUMMARY OF THE INVENTION

We have discovered that a combination of a non-steroidal immunophilin-dependent immunosuppressant (NsIDI) (e.g., cyclosporine A) and a non-steroidal immunophilin-dependent immunosuppressant enhancer (NsIDIE) (e.g., a selective serotonin reuptake inhibitor (SSR₁), a tricyclic antidepressant, a phenoxy phenol, an antihistamine, a phenothiazine, or a mu opioid receptor agonist) is more effective in suppressing secretion of proinflammatory cytokines than either agent alone. Thus, combinations of an NsIDI and an NsIDIE, as well as their structural or functional analogs, can be used in an anti-immunoinflammatory combination of the invention.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

In one aspect, the invention generally features a composition containing a non-steroidal immunophilin-dependent immunosuppressant (NsIDI) and an NsIDI enhancer (NsIDIE) in amounts that together are sufficient in vivo to decrease proinflammatory cytokine secretion or production or to treat an immunoinflammatory disorder.

Optionally, the composition further contains a non-steroidal anti-inflammatory drug (NSAID), a COX-2 inhibitor, a biologic, a disease-modifying anti-rheumatic drugs (DMARD), a xanthine, an anticholinergic compound, a beta receptor agonist, a bronchodilator, a non-steroidal calcineurin inhibitor, a vitamin D analog, a psoralen, a retinoid, or a 5-amino salicylic acid. In some embodiments, the composition is formulated for topical or systemic administration.

The invention also provides a method of decreasing proinflammatory cytokine secretion or production in a patient, the method includes administering to the patient a composition containing a non-steroidal immunophilin-dependent immunosuppressant (NsIDI) and an NsIDI enhancer (NsIDIE) simultaneously or within 14 days of each other in amounts sufficient in vivo to decrease proinflammatory cytokine secretion or production in the patient.

The invention also features a method of decreasing proinflammatory cytokine secretion or production in a patient. The method includes administering to the patient an NsIDI and an NsIDIE simultaneously or within 14 days of each other in amounts sufficient in vivo to decrease proinflammatory cytokine secretion or production in the patient.

In addition, the invention features a method for treating a patient diagnosed with or at risk of developing an immunoinflammatory disorder. The method includes administering to the patient an NsIDI and an NsIDIE simultaneously or within 14 days of each other in amounts sufficient to treat the patient.

The invention also features a method of decreasing proinflammatory cytokine secretion or production in a cell (e.g., a mammalian cell in vivo). The method includes contacting the cell with an NsIDI and an NsIDIE simultaneously or within 14 days of each other in amounts sufficient in vivo to decrease proinflammatory cytokine secretion or production in the cell.

The invention further provides a kit containing a composition containing an NsIDI and an NsIDIE; and instructions for administering the composition to a patient diagnosed with or at risk of developing an immunoinflammatory disorder.

The invention also provides a kit containing an NsIDI, an NsIDIE; and instructions for administering the NsIDI and the NsIDIE to a patient diagnosed with or at risk of developing an immunoinflammatory disorder.

The invention also provides a kit containing an NsIDI; and instructions for administering the NsIDI and an NsIDIE to a patient diagnosed with or at risk of developing an immunoinflammatory disorder.

In addition, the invention provides a kit containing an NsIDIE and instructions for administering the NsIDIE and an NsIDI to a patient diagnosed with or at risk of developing an immunoinflammatory disorder.

The invention also features a method for identifying combinations of compounds useful for suppressing the secretion of proinflammatory cytokines in a patient in need of such treatment. The method includes contacting cells in vitro with an NsIDI and a candidate compound; and (b) determining whether the combination of the NsIDI and the candidate compound reduces cytokine levels in blood cells stimulated to secrete the cytokines relative to cells contacted with the NsIDI but not contacted with the candidate compound or cells contacted with the candidate compound but not with the NsIDI, wherein a reduction of the cytokine levels identifies the combination as a combination that is useful for treating a patient in need of such treatment.

In preferred embodiments of any of the previous aspects, an NsIDI is, for example, a calcineurin inhibitor, such as cyclosporine, tacrolimus, ascomycin, pimecrolimus, or ISAtx247, or an FK506-binding protein, such as rapamycin or everolimus.

In preferred embodiments of any of the previous aspects, an NsIDI enhancer (NsIDIE) is, for example, a selective serotonin reuptake inhibitor (SSR₁), a tricyclic antidepressant (TCA), a phenoxy phenol, an antihistamine, a phenothiazine, or a mu opioid receptor agonist.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “non-steroidal immunophilin-dependent immunosuppressant enhancer” or “NsIDIE” is meant any compound that increases the efficacy of a non-steroidal immunophilin-dependent immunosuppressant. NsIDIEs include selective serotonin reuptake inhibitors, tricyclic antidepressants, phenoxy phenols (e.g., triclosan), antihistamines, phenothiazines, and mu opioid receptor agonists.

By “antihistamine” is meant a compound that blocks the action of histamine. Classes of antihistamines include, but are not limited to, ethanolamines, ethylenediamine, phenothiazine, alkylamines, piperazines, and piperidines.

By “selective serotonin reuptake inhibitor” or “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are described herein.

By “tricyclic antidepressant” or “TCA” is meant a compound having one of the formulas (I), (II), (III), or (IV):

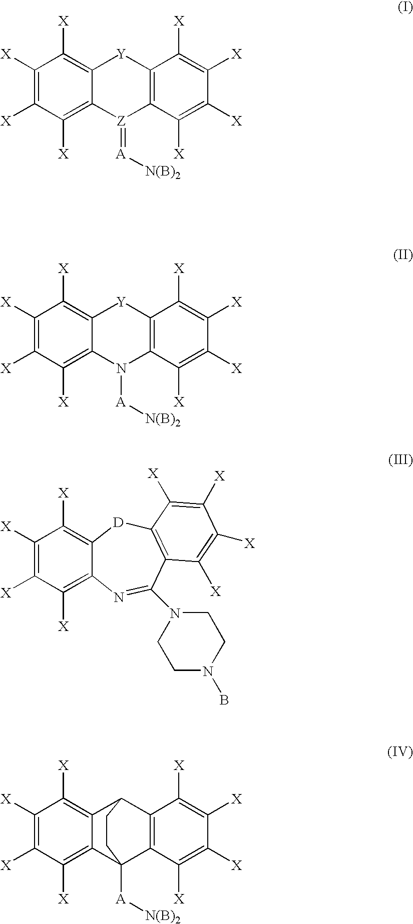

wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or mono-unsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, S(O)₀₋₂.

In preferred embodiments, each X is, independently, H, Cl, or F; Y is (CH₂)₂, Z is C; A is (CH₂)₃; and each B is, independently, H, Cl, or F.

Exemplary tricyclic antidepressants are maprotiline, amoxapine, 8-hydroxyamoxapine, 7-hydroxyamoxapine, loxapine, loxapine succinate, loxapine hydrochloride, 8-hydroxyloxapine, amitriptyline, clomipramine, doxepin, imipramine, trimipramine, desipramine, nortriptyline, and protriptyline.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated. Examples of corticosteroids are provided herein.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant the dosage between the low dosage and the high dosage.

By “treating” is meant administering or prescribing a pharmaceutical composition for the treatment or prevention of an immunoinflammatory disease.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In one embodiment of the invention, the patient subject to a treatment employing an SSRI or a TCA described herein does not have clinical depression, an anxiety or panic disorder, an obsessive/compulsive disorder, alcoholism, an eating disorder, an attention-deficit disorder, a borderline personality disorder, a sleep disorder, a headache, premenstrual syndrome, an irregular heartbeat, schizophrenia, Tourette's syndrome, or phobias.

By “an amount sufficient” is meant the amount of a compound in the methods, compositions, and kits of the invention, required to treat or prevent an immunoinflammatory disease in a clinically relevant manner. A sufficient amount of an active compound used to practice the present invention for therapeutic treatment of conditions caused by or contributing to an immunoinflammatory disease varies depending upon the manner of administration, the age, body weight, and general health of the patient. Ultimately, the prescribers will decide the appropriate amount and dosage regimen.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hepatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; focal segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; immune thrombocytopenic purpura inflammatory bowel or gastrointestinal disorders, inflammatory dermatoses; lichen planus; lupus nephritis; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

“Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both a proliferative skin disease and an inflammatory dermatosis. An example of such a disease is psoriasis.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

By “acyl” is meant a chemical moiety with the formula R—C(O)—, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “amido” is meant a chemical substituent of the formula —NRR′, wherein the nitrogen atom is part of an amide bond (e.g., —C(O)—NRR′) and wherein R and R′ are each, independently, selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, and C₁₋₇ heteroalkyl, or —NRR′ forms a C₂₋₆ heterocyclyl ring, as defined above, but containing at least one nitrogen atom, such as piperidino, morpholino, and azabicyclo, among others.

By “halide” or “halo” is meant bromine, chlorine, iodine, or fluorine.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “paroxetine” is meant the free base, as well as any pharmaceutically acceptable salt thereof (e.g., paroxetine maleate, paroxetine hydrochloride hemihydrate, and paroxetine mesylate).

Other features and advantages of the invention will be apparent from the following detailed description, and from the claims.

### DETAILED DESCRIPTION

The invention features methods, compositions, and kits for the administration of an effective amount of a non-steroidal immunophilin-dependent immunosuppressant (NsIDI), such as cyclosporine, and a non-steroidal immunophilin-dependent immunosuppressant enhancer (NsIDIE), e.g., a selective serotonin reuptake inhibitor, a tricyclic antidepressant, a phenoxy phenol, an antihistamine, a phenothiazine, or a mu opioid receptor agonist.

The invention is described in greater detail below.

Non-Steroidal Immunophilin-Dependent Immunosuppressants

In one embodiment, the invention features methods, compositions, and kits employing an NsIDI and an NsIDIE, optionally with a corticosteroid or other agent described herein.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

Cyclosporines

The cyclosporines are fungal metabolites that comprise a class of cyclic oligopeptides that act as immunosuppressants. Cyclosporine A, and its deuterated analogue ISAtx247, are hydrophobic cyclic polypeptide consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH—Cs (209-825), Allo-Thr-2Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL™).

Typically, patient dosage of an oral cyclosporine varies according to the patient's condition, but some standard recommended dosages in prior art treatment regimens are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids.

Additional information is provided in Table 1.

TABLE 1
NsIDIs

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 | 0.5-4 | 6-8 | 6-8 | ˜7-12 | 2.2-6.0 |
| (NEORAL) | | mg/kg/day | mg/kg/day | mg/kg/day | mg/kg/day | mg/kg/day | mg/kg/day |
| | | | | (oral-fistulizing) | (oral) | | |
| Tacrolimus | .03-0.1% | .05-1.15 | 1-3 | 0.1-0.2 | 0.1-0.2 | 0.1-0.2 | N/A |
| | cream/twice | mg/kg/day | mg/day | mg/kg/day | mg/kg/day | mg/kg/day | |
| | day (30 and | (oral) | (oral) | (oral) | (oral) | (oral) | |
| | 60 gram | | | | | | |
| | tubes) | | | | | | |
| Pimecrolimus | 1% | 40-60 | 40-60 | 80-160 | 160-240 | 40-120 | 40-120 |
| | cream/twice | mg/day | mg/day | mg/day | mg/day | mg/day | mg/day |
| | day (15, 30, | (oral) | (oral) | (oral) | (oral) | (oral) | (oral) |
| | 100 gram | | | | | | |
| | tubes) | | | | | | |

Legend
CsA = cyclosporine A
RA = rheumatoid arthritis
UC = ulcerative colitis
SLE = systemic lupus erythamatosus

Tacrolimus

Tacrolimus (PROGRAF, Fujisawa), also known as FK506, is an immunosuppressive agent that targets T-cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages used in prior art treatment regimens are provided below. Patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is an 33-epi-chloro derivative of the ascomycin. It is produced by the strain Streptomyces hygroscopicus var. ascomyceitus. Like tacrolimus, pimecrolimus (ELIDEL™ Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is particularly useful for the treatment of atopic dermatitis. Pimecrolimus is currently available as a 1% cream. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin (RAPAMUNE® sirolimus, Wyeth) is a cyclic lactone produced by Steptomyces hygroscopicus. Rapamycin is an immunosuppressive agent that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A.

Rapamycin is currently available for oral administration in liquid and tablet formulations. RAPAMUNE™ liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1-mg/m²/day maintenance dose is used.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcineurin inhibitors, these agents are useful in the methods of the invention.

Selective Serotonin Reuptake Inhibitors

In one embodiment, the methods, compositions, and kits of the invention employ a selective serotonin reuptake inhibitor (SSR₁), or a structural or functional analog thereof in combination with a non-steroidal immunophilin-dependent immunosuppressant (NsIDI). Suitable SSRIs include cericlamine (e.g., cericlamine hydrochloride); citalopram (e.g., citalopram hydrobromide); clovoxamine; cyanodothiepin; dapoxetine; escitalopram (escitalopram oxalate); femoxetine (e.g., femoxetine hydrochloride); fluoxetine (e.g., fluoxetine hydrochloride); fluvoxamine (e.g., fluvoxamine maleate); ifoxetine; indalpine (e.g., indalpine hydrochloride); indeloxazine (e.g., indeloxazine hydrochloride); litoxetine; milnacipran (e.g., minlacipran hydrochloride); paroxetine (e.g., paroxetine hydrochloride hemihydrate; paroxetine maleate; paroxetine mesylate); sertraline (e.g., sertraline hydrochloride); sibutramine, tametraline hydrochloride; viqualine; and zimeldine (e.g., zimeldine hydrochloride).

SSRIs are drugs that inhibit 5-hydroxytryptamine (5-HT) uptake by neurons of the central nervous system. SSRIs show selectivity with respect to 5-HT over norepinephrine uptake. They are less likely than tricyclic antidepressants to cause anticholinergic side effects and are less dangerous in overdose. SSRIs, such as paroxetine, sertraline, fluoxetine, citalopram, fluvoxamine, nor₁-citalopram, venlafaxine, milnacipran, nor₂-citalopram, nor-fluoxetine, or nor-sertraline are used to treat a variety of psychiatric disorders, including depression, anxiety disorders, panic attacks, and obsessive-compulsive disorder. Dosages given here are the standard recommended doses for psychiatric disorders. In practicing the methods of the invention, effective amounts may be different.

Administration of each drug in the combination can, independently, be one to four times daily for one day to one year, and may even be for the life of the patient. Chronic, long-term administration will be indicated in many cases. Typically, patient dosage of an SSRI varies according to the patient's condition. SSRIs may be administered orally, by suppository, or by injection. Often doses are provided orally once a day as a tablet or a liquid concentrate.

Cericlamine

Cericlamine has the following structure:

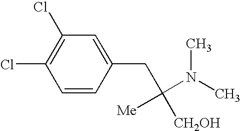

Structural analogs of cericlamine are those having the formula:

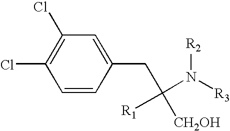

as well as pharmaceutically acceptable salts thereof, wherein R₁ is a C₁-C₄ alkyl and R₂ is H or C₁₋₄ alkyl, R₃ is H, C₁₋₄ alkyl, C₂₋₄ alkenyl, phenylalkyl or cycloalkylalkyl with 3 to 6 cyclic carbon atoms, alkanoyl, phenylalkanoyl or cycloalkylcarbonyl having 3 to 6 cyclic carbon atoms, or R₂ and R₃ form, together with the nitrogen atom to which they are linked, a heterocycle saturated with 5 to 7 chain links which can have, as the second heteroatom not directly connected to the nitrogen atom, an oxygen, a sulphur or a nitrogen, the latter nitrogen heteroatom possibly carrying a C₂₋₄ alkyl.

Exemplary cericlamine structural analogs are 2-methyl-2-amino-3-(3,4-dichlorophenyl)-propanol, 2-pentyl-2-amino-3-(3,4-dichlorophenyl)-propanol, 2-methyl-2-methylamino-3-(3,4-dichlorophenyl)-propanol, 2-methyl-2-dimethylamino-3-(3,4-dichlorophenyl)-propanol, and pharmaceutically acceptable salts of any thereof.

Citalopram

Citalopram HBr (CELEXA™) is a racemic bicyclic phthalane derivative designated (±)-1-(3-dimethylaminopropyl)-1-(4-fluorophenyl)-1,3-dihydroisobenzofuran-5-carbonitrile, HBr. Citalopram undergoes extensive metabolization; nor₁-citalopram and nor₂-citalopram are the main metabolites. Citalopram is available in 10 mg, 20 mg, and 40 mg tablets for oral administration. CELEXA™ oral solution contains citalopram HBr equivalent to 2 mg/mL citalopram base. CELEXA™ is typically administered at an initial dose of 20 mg once daily, generally with an increase to a dose of 40 mg/day. Dose increases typically occur in increments of 20 mg at intervals of no less than one week.

Citalopram has the following structure:

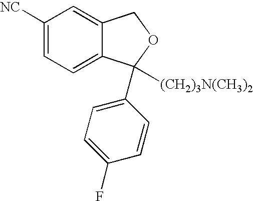

Structural analogs of citalopram are those having the formula:

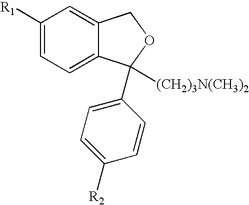

as well as pharmaceutically acceptable salts thereof, wherein each of R₁ and R₂ is independently selected from the group consisting of bromo, chloro, fluoro, trifluoromethyl, cyano and R—CO—, wherein R is C₁₋₄ alkyl.

Exemplary citalopram structural analogs (which are thus SSRI structural analogs according to the invention) are 1-(4′-fluorophenyl)-1-(3-dimethylaminopropyl)-5-bromophthalane; 1-(4′-chlorophenyl)-1-(3-dimethylaminopropyl)-5-chlorophthalane; 1-(4′-bromophenyl)-1-(3-dimethylaminopropyl)-5-chlorophthalane; 1-(4′-fluorophenyl)-1-(3-dimethylaminopropyl)-5-chlorophthalane; 1-(4′-chlorophenyl)-1-(3-dimethylaminopropyl)-5-trifluoromethyl-phthalane; 1-(4′-bromophenyl)-1-(3-dimethylaminopropyl)-5-trifluoromethyl-phthalane; 1-(4′-fluorophenyl)-1-(3-dimethylaminopropyl)-5-trifluoromethyl-phthalane; 1-(4′-fluorophenyl)-1-(3-dimethylaminopropyl)-5-fluorophthalane; 1-(4′-chlorophenyl)-1-(3-dimethylaminopropyl)-5-fluorophthalane; 1-(4′-chlorophenyl)-1-(3-dimethylaminopropyl)-5-phthalancarbonitrile; 1-(4′-fluorophenyl)-1-(3-dimethylaminopropyl)-5-phthalancarbonitrile; 1-(4′-cyanophenyl)-1-(3-dimethylaminopropyl)-5-phthalancarbonitrile; 1-(4′-cyanophenyl)-1-(3-dimethylaminopropyl)-5-chlorophthalane; 1-(4′-cyanophenyl)-1-(3-dimethylaminopropyl)-5-trifluoromethylphthalane; 1-(4′-fluorophenyl)-1-(3-dimethylaminopropyl)-5-phthalancarbonitrile; 1-(4′-chlorophenyl)-1-(3-dimethylaminopropyl)-5-ionylphthalane; 1-(4-(chlorophenyl)-1-(3-dimethylaminopropyl)-5-propionylphthalane; and pharmaceutically acceptable salts of any thereof.

Clovoxamine

Clovoxamine has the following structure:

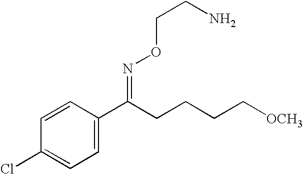

Structural analogs of clovoxamine are those having the formula:

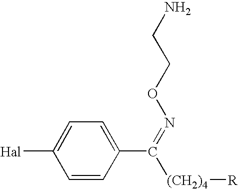

as well as pharmaceutically acceptable salts thereof, wherein Hal is a chloro, bromo, or fluoro group and R is a cyano, methoxy, ethoxy, methoxymethyl, ethoxymethyl, methoxyethoxy, or cyanomethyl group.

Exemplary clovoxamine structural analogs are 4′-chloro-5-ethoxyvalerophenone O-(2-aminoethyl)oxime; 4′-chloro-5-(2-methoxyethoxy)valerophenone O-(2-aminoethyl)oxime; 4′-chloro-6-methoxycaprophenone O-(2-aminoethyl)oxime; 4′-chloro-6-ethoxycaprophenone O-(2-aminoethyl)oxime; 4′-bromo-5-(2-methoxyethoxy)valerophenone O-(2-aminoethyl)oxime; 4′-bromo-5-methoxyvalerophenone O-(2-aminoethyl)oxime; 4′-chloro-6-cyanocaprophenone O-(2-aminoethyl)oxime; 4′-chloro-5-cyanovalerophenone O-(2-aminoethyl)oxime; 4′-bromo-5-cyanovalerophenone 0-(2-aminoethyl)oxime; and pharmaceutically acceptable salts of any thereof.

Femoxetine

Femoxetine has the following structure:

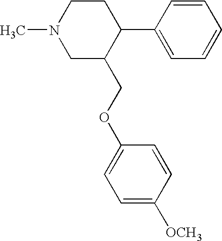

Structural analogs of femoxetine are those having the formula:

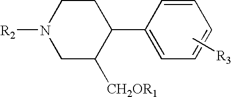

wherein R₁ represents a C₁₋₄ alkyl or C₂₋₄ alkynyl group, or a phenyl group optionally substituted by C₁₋₄ alkyl, C₁₋₄ alkylthio, C₁₋₄ alkoxy, bromo, chloro, fluoro, nitro, acylamino, methylsulfonyl, methylenedioxy, or tetrahydronaphthyl, R₂ represents a C₁₋₄ alkyl or C₂₋₄ alkynyl group, and R₃ represents hydrogen, C₁₋₄ alkyl, C₁₋₄alkoxy, trifluoroalkyl, hydroxy, bromo, chloro, fluoro, methylthio, or aralkyloxy.

Exemplary femoxetine structural analogs are disclosed in Examples 7-67 of U.S. Pat. No. 3,912,743, hereby incorporated by reference.

Fluoxetine

Fluoxetine hydrochloride ((±)—N-methyl-3-phenyl-3-[((alpha),(alpha),(alpha)-trifluoro-p-tolyl)oxy]propylamine hydrochloride) is sold as PROZAC™ in 10 mg, 20 mg, and 40 mg tablets for oral administration. The main metabolite of fluoxetine is nor-fluoxetine. Fluoxetine hydrochloride may also be administered as an oral solution equivalent to 20 mg/5 mL of fluoxetine. A delayed release formulation contains enteric-coated pellets of fluoxetine hydrochloride equivalent to 90 mg of fluoxetine. A dose of 20 mg/day, administered in the morning, is typically recommended as the initial dose. A dose increase may be considered after several weeks if no clinical improvement is observed. Doses above 20 mg/day may be administered on a once a day (morning) or twice a day schedule (e.g., morning and noon) and should not exceed a maximum dose of 80 mg/day.

Fluoxetine has the following structure:

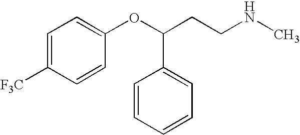

Structural analogs of fluoxetine are those compounds having the formula:

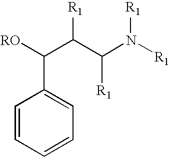

as well as pharmaceutically acceptable salts thereof, wherein each R₁ is independently hydrogen or methyl; R is naphthyl or

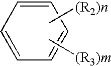

wherein each of R₂ and R₃ is, independently, bromo, chloro, fluoro, trifluoromethyl, C₁₋₄ alkyl, C₁₋₃ alkoxy or C₃₋₄ alkenyl; and each of n and m is, independently, 0, 1 or 2. When R is naphthyl, it can be either α-naphthyl or β-naphthyl.

Exemplary fluoxetine structural analogs are 3-(p-isopropoxyphenoxy)-3-phenylpropylamine methanesulfonate, N,N-dimethyl 3-(3′,4′-dimethoxyphenoxy)-3-phenylpropylamine p-hydroxybenzoate, N,N-dimethyl 3-(α-naphthoxy)-3-phenylpropylamine bromide, N,N-dimethyl 3-(β-naphthoxy)-3-phenyl-1-methylpropylamine iodide, 3-(2′-methyl-4′,5′-dichlorophenoxy)-3-phenylpropylamine nitrate, 3-(p-t-butylphenoxy)-3-phenylpropylamine glutarate, N-methyl 3-(2′-chloro-p-tolyloxy)-3-phenyl-1-methylpropylamine lactate, 3-(2′,4′-dichlorophenoxy)-3-phenyl-2-methylpropylamine citrate, N,N-dimethyl 3-(m-anisyloxy)-3-phenyl-1-methylpropylamine maleate, N-methyl 3-(p-tolyloxy)-3-phenylpropylamine sulfate, N,N-dimethyl 3-(2′,4′-difluorophenoxy)-3-phenylpropylamine 2,4-dinitrobenzoate, 3-(o-ethylphenoxy)-3-phenylpropylamine dihydrogen phosphate, N-methyl 3-(2′-chloro-4′-isopropylphenoxy)-3-phenyl-2-methylpropylamine maleate, N,N-dimethyl 3-(2′-alkyl-4′-fluorophenoxy)-3-phenyl-propylamine succinate, N,N-dimethyl 3-(o-isopropoxyphenoxy)-3-phenyl-propylamine phenylacetate, N,N-dimethyl 3-(o-bromophenoxy)-3-phenyl-propylamine β-phenylpropionate, N-methyl 3-(p-iodophenoxy)-3-phenyl-propylamine propiolate, and N-methyl 3-(3-n-propylphenoxy)-3-phenyl-propylamine decanoate.

Fluvoxamine

Fluvoxamine maleate (LUVOX™) is chemically designated as 5-methoxy-4′-(trifluoromethyl) valerophenone (E)-O-(2-aminoethyl)oxime maleate. Fluvoxamine maleate is supplied as 50 mg and 100 mg tablets. Treatment is typically initiated at 50 mg given once daily at bedtime, and then increased to 100 mg daily at bedtime after a few days, as tolerated. The effective daily dose usually lies between 100 and 200 mg, but may be administered up to a maximum of 300 mg.

Fluvoxamine has the following structure:

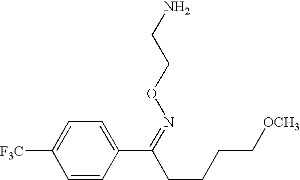

Structural analogs of fluvoxamine are those having the formula:

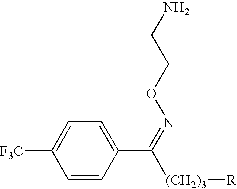

as well as pharmaceutically acceptable salts thereof, wherein R is cyano, cyanomethyl, methoxymethyl, or ethoxymethyl.

Indalpine

Indalpine has the following structure:

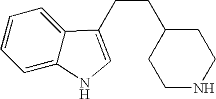

Structural analogs of indalpine are those having the formula:

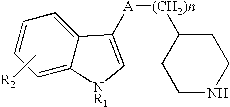

or pharmaceutically acceptable salts thereof, wherein R₁ is a hydrogen atom, a C₁-C₄ alkyl group, or an aralkyl group of which the alkyl has 1 or 2 carbon atoms, R₂ is hydrogen, C₁₋₄ alkyl, C₁₋₄ alkoxy or C₁₋₄ alkylthio, chloro, bromo, fluoro, trifluoromethyl, nitro, hydroxy, or amino, the latter optionally substituted by one or two C₁₋₄ alkyl groups, an acyl group or a C₁₋₄alkylsulfonyl group; A represents —CO or —CH₂— group; and n is 0, 1 or 2.

Exemplary indalpine structural analogs are indolyl-3 (piperidyl-4 methyl) ketone; (methoxy-5-indolyl-3) (piperidyl-4 methyl) ketone; (chloro-5-indolyl-3) (piperidyl-4 methyl) ketone; (indolyl-3)-1(piperidyl-4)-3 propanone, indolyl-3 piperidyl-4 ketone; (methyl-1 indolyl-3) (piperidyl-4 methyl) ketone, (benzyl-1 indolyl-3) (piperidyl-4 methyl) ketone; [(methoxy-5 indolyl-3)-2 ethyl]-piperidine, [(methyl-1 indolyl-3)-2 ethyl]-4-piperidine; [(indolyl-3)-2 ethyl]-4 piperidine; (indolyl-3 methyl)-4 piperidine, [(chloro-5 indolyl-3)-2 ethyl]-4 piperidine; [(indolyl-b 3)-3 propyl]-4 piperidine; [(benzyl-1 indolyl-3)-2 ethyl]-4 piperidine; and pharmaceutically acceptable salts of any thereof.

Indeloxazine

Indeloxezine has the following structure:

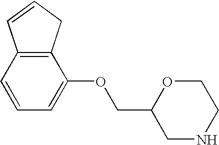

Structural analogs of indeloxazine are those having the formula:

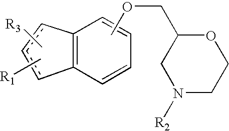

and pharmaceutically acceptable salts thereof, wherein R₁ and R₃ each represents hydrogen, C₁₋₄ alkyl, or phenyl; R₂ represents hydrogen, C₁₋₄ alkyl, C₄₋₇ cycloalkyl, phenyl, or benzyl; one of the dotted lines means a single bond and the other means a double bond, or the tautomeric mixtures thereof.

Exemplary indeloxazine structural analogs are 2-(7-indenyloxymethyl)-4-isopropylmorpholine; 4-butyl-2-(7-indenyloxymethyl)morpholine; 2-(7-indenyloxymethyl)-4-methylmorpholine; 4-ethyl-2-(7-indenyloxymethyl)morpholine, 2-(7-indenyloxymethyl)-morpholine; 2-(7-indenyloxymethyl)-4-propylmorpholine; 4-cyclohexyl-2-(7-indenyloxymethyl)morpholine; 4-benzyl-2-(7-indenyloxymethyl)-morpholine; 2-(7-indenyloxymethyl)-4-phenylmorpholine; 2-(4-indenyloxymethyl)morpholine; 2-(3-methyl-7-indenyloxymethyl)-morpholine; 4-isopropyl-2-(3-methyl-7-indenyloxymethyl)morpholine; 4-isopropyl-2-(3-methyl-4-indenyloxymethyl)morpholine; 4-isopropyl-2-(3-methyl-5-indenyloxymethyl)morpholine; 4-isopropyl-2-(1-methyl-3-phenyl-6-indenyloxymethyl)morpholine; 2-(5-indenyloxymethyl)-4-isopropyl-morpholine, 2-(6-indenyloxymethyl)-4-isopropylmorpholine; and 4-isopropyl-2-(3-phenyl-6-indenyloxymethyl)morpholine; as well as pharmaceutically acceptable salts of any thereof.

Milnacipram

Milnacipran (IXEL™, Cypress Bioscience Inc.) has the chemical formula (Z)-1-diethylaminocarbonyl-2-aminoethyl-1-phenyl-cyclopropane)hydrochlorate, and is provided in 25 mg and 50 mg tablets for oral administration. It is typically administered in dosages of 25 mg once a day, 25 mg twice a day, or 50 mg twice a day for the treatment of severe depression.

Milnacipram has the following structure:

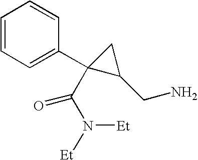

Structural analogs of milnacipram are those having the formula:

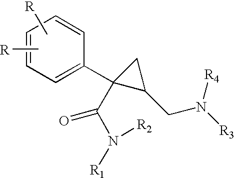

as well as pharmaceutically acceptable salts thereof, wherein each R, independently, represents hydrogen, bromo, chloro, fluoro, C₁₋₄ alkyl, C₁₋₄ alkoxy, hydroxy, nitro or amino; each of R₁ and R₂, independently, represents hydrogen, C₁₋₄ alkyl, C₆₋₁₂ aryl or C₇₋₁₄ alkylaryl, optionally substituted, preferably in para position, by bromo, chloro, or fluoro, or R₁ and R₂ together form a heterocycle having 5 or 6 members with the adjacent nitrogen atoms; R₃ and R₄ represent hydrogen or a C₁₋₄ alkyl group or R₃ and R₄ form with the adjacent nitrogen atom a heterocycle having 5 or 6 members, optionally containing an additional heteroatom selected from nitrogen, sulphur, and oxygen.

Exemplary milnacipram structural analogs are 1-phenyl 1-aminocarbonyl 2-dimethylaminomethyl cyclopropane; 1-phenyl 1-dimethylaminocarbonyl 2-dimethylaminomethyl cyclopropane; 1-phenyl 1-ethylaminocarbonyl 2-dimethylaminomethyl cyclopropane; 1-phenyl 1-diethylaminocarbonyl 2-aminomethyl cyclopropane; 1-phenyl 2-dimethylaminomethyl N-(4′-chlorophenyl)cyclopropane carboxamide; 1-phenyl 2-dimethylaminomethyl N-(4′-chlorobenzyl)cyclopropane carboxamide; 1-phenyl 2-dimethylaminomethyl N-(2-phenylethyl)cyclopropane carboxamide; (3,4-dichloro-1-phenyl) 2-dimethylaminomethyl N,N-dimethylcyclopropane carboxamide; 1-phenyl 1-pyrrolidinocarbonyl 2-morpholinomethyl cyclopropane; 1-p-chlorophenyl 1-aminocarbonyl 2-aminomethyl cyclopropane; 1-orthochlorophenyl 1-aminocarbonyl 2-dimethylaminomethyl cyclopropane; 1-p-hydroxyphenyl 1-aminocarbonyl 2-dimethylaminomethyl cyclopropane; 1-p-nitrophenyl 1-dimethylaminocarbonyl 2-dimethylaminomethyl cyclopropane; 1-p-aminophenyl 1-dimethylaminocarbonyl 2-dimethylaminomethyl cyclopropane; 1-p-tolyl 1-methylaminocarbonyl 2-dimethylaminomethyl cyclopropane; 1-p-methoxyphenyl 1-aminomethylcarbonyl 2-aminomethyl cyclopropane; and pharmaceutically acceptable salts of any thereof.

Paroxetine

Paroxetine hydrochloride ((−)-trans-4R-(4′-fluorophenyl)-3S-[(3′,4′-methylenedioxyphenoxy)methyl]piperidine hydrochloride hemihydrate) is provided as PAXIL™. Controlled-release tablets contain paroxetine hydrochloride equivalent to paroxetine in 12.5 mg, 25 mg, or 37.5 mg dosages. One layer of the tablet consists of a degradable barrier layer and the other contains the active material in a hydrophilic matrix. The recommended initial dose of PAXIL™ is 25 mg/day. Some patients not responding to a 25 mg dose may benefit from dose increases, in 12.5 mg/day increments, up to a maximum of 62.5 mg/day. Dose changes typically occur at intervals of at least one week.

Paroxetine has the following structure:

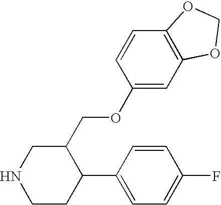

Structural analogs of paroxetine are those having the formula:

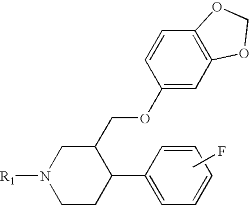

and pharmaceutically acceptable salts thereof, wherein R₁ represents hydrogen or a C₁₋₄ alkyl group, and the fluorine atom may be in any of the available positions.

Sertraline

Sertraline ((1S-cis)-4-(3,4-dichlorophenyl)-1,2,3,4-tetrahydro-N-methyl-1-nanphthalenamine hydrochloride) is provided as ZOLOFT™ in 25 mg, 50 mg and 100 mg tablets for oral administration. Because sertraline undergoes extensive metabolic transformation into a number of metabolites that may be therapeutically active, these metabolites may be substituted for sertraline in an anti-inflammatory combination of the invention. The metabolism of sertraline includes, for example, oxidative N-demethylation to yield N-desmethylsertraline (nor-sertraline). ZOLOFT is typically administered at a dose of 50 mg once daily.

Sertraline has the following structure:

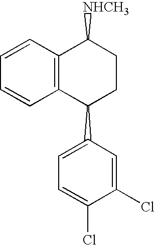

Structural analogs of sertraline are those having the formula:

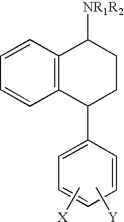

wherein R₁ is selected from the group consisting of hydrogen and C₁₋₄ alkyl; R₂ is C₁₋₄ alkyl; X and Y are each selected from the group consisting of hydrogen, fluoro, chloro, bromo, trifluoromethyl, C₁₋₃ alkoxy, and cyano; and W is selected from the group consisting of hydrogen, fluoro, chloro, bromo, trifluoromethyl and C₁₋₃ alkoxy. Preferred sertraline analogs are in the cis-isomeric configuration. The term “cis-isomeric” refers to the relative orientation of the NR₁R₂ and phenyl moieties on the cyclohexene ring (i.e. they are both oriented on the same side of the ring). Because both the 1- and 4-carbons are asymmetrically substituted, each cis-compound has two optically active enantiomeric forms denoted (with reference to the 1-carbon) as the cis-(1R) and cis-(1S) enantiomers.

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

Sibutramine Hydrochloride Monohydrate

Sibutramine hydrochloride monohydrate (MERIDIA™) is an orally administered agent for the treatment of obesity. Sibutramine hydrochloride is a racemic mixture of the (+) and (−) enantiomers of cyclobutanemethanamine, 1-(4-chlorophenyl)-N,N-dimethyl-(alpha)-(2-methylpropyl)-, hydrochloride, monohydrate. Each MERIDIA™ capsule contains 5 mg, 10 mg, or 15 mg of sibutramine hydrochloride monohydrate. The recommended starting dose of MERIDIA™ is 10 mg administered once daily with or without food. If there is inadequate weight loss, the dose may be titrated after four weeks to a total of 15 mg once daily. The 5 mg dose is typically reserved for patients who do not tolerate the 10 mg dose.

Zimeldine

Zimeldine has the following structure:

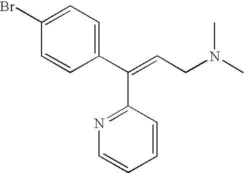

Structural analogs of zimeldine are those compounds having the formula:

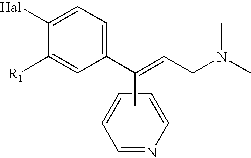

and pharmaceutically acceptable salts thereof, wherein the pyridine nucleus is bound in ortho-, meta- or para-position to the adjacent carbon atom and where R₁ is selected from the group consisting of H, chloro, fluoro, and bromo.

Exemplary zimeldine analogs are (e)- and (z)-3-(4′-bromophenyl-3-(2″-pyridyl)-dimethylallylamine; 3-(4′-bromophenyl)-3-(3″-pyridyl)-dimethylallylamine; 3-(4′-bromophenyl)-3-(4″-pyridyl)-dimethylallylamine; and pharmaceutically acceptable salts of any thereof.

Structural analogs of any of the above SSRIs are considered herein to be SSRI analogs and thus may be employed in any of the methods, compositions, and kits of the invention.

Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

Analogs

Functional analogs of SSRIs can also be used in the methods, compositions, and kits of the invention. Exemplary SSRI functional analogs are provided below. One class of SSRI analogs are SNRIs (selective serotonin norepinephrine reuptake inhibitors), which include venlafaxine and duloxetine.

Venlafaxine

Venlafaxine hydrochloride (EFFEXOR™) is an antidepressant for oral administration. It is designated (R/S)-1-[2-(dimethylamino)-1-(4-methoxyphenyl)ethyl]cyclohexanol hydrochloride or (±)-1-[(alpha)-[(dimethyl-amino)methyl]-p-methoxybenzyl]cyclohexanol hydrochloride. Compressed tablets contain venlafaxine hydrochloride equivalent to 25 mg, 37.5 mg, 50 mg, 75 mg, or 100 mg venlafaxine. The recommended starting dose for venlafaxine is 75 mg/day, administered in two or three divided doses, taken with food. Depending on tolerability and the need for further clinical effect, the dose may be increased to 150 mg/day. If desirable, the dose can be further increased up to 225 mg/day. When increasing the dose, increments of up to 75 mg/day are typically made at intervals of no less than four days.

Venlafaxine has the following structure:

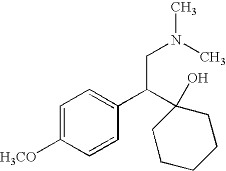

Structural analogs of venlafaxine are those compounds having the formula:

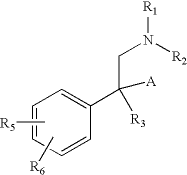

as well as pharmaceutically acceptable salts thereof, wherein A is a moiety of the formula:

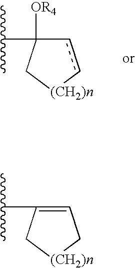

where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Duloxetine

Duloxetine has the following structure:

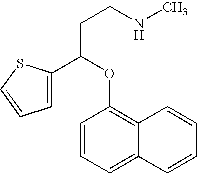

Structural analogs of duloxetine are those compounds described by the formula disclosed in U.S. Pat. No. 4,956,388, hereby incorporated by reference.

Other SSRI analogs are 4-(2-fluorophenyl)-6-methyl-2-piperazinothieno [2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO04/004734.

SSRI Standard Recommended Dosages

Standard recommended dosages for exemplary SSRIs are provided in Table 2, below. Other standard dosages are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002).

TABLE 2

| Compound | Standard Dose | |
|---|---|---|
| Fluoxetine | 20-80 | mg/day |
| Sertraline | 50-200 | mg/day |
| Paroxetine | 20-50 | mg/day |
| Fluvoxamine | 50-300 | mg/day |
| Citalopram | 10-80 | mg qid |
| Escitalopram | 10 | mg qid |

Tricyclic Antidepressants

In another embodiment, the methods, compositions, and kits of the invention employ tricyclic antidepressant (TCA), or a structural or functional analog thereof in combination with a non-steroidal immunophilin-dependent immunosuppressant (NsIDI). Maprotiline (brand name LUDIOMIL) is a secondary amine tricyclic antidepressant that inhibits norepinephrine reuptake and is structurally related to imipramine, a dibenzazepine. While such agents have been used for the treatment of anxiety and depression, we report herein that maprotiline increases the potency of an immunosuppressive agent, and is useful in an anti-inflammatory combination of the invention.

Maprotiline (brand name LUDIOMIL) and maprotiline structural analogs have three-ring molecular cores (see formula (IV), supra). These analogs include other tricyclic antidepressants (TCAs) having secondary amine side chains (e.g., nortriptyline, protriptyline, desipramine) as well as N-demethylated metabolites of TCAs having tertiary amine side chains. Preferred maprotiline structural and functional analogs include tricyclic antidepressants that are selective inhibitors of norepinephrine reuptake. Tricyclic compounds that can be used in the methods, compositions, and kits of the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-1′-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 11-(4-methyl-1-piperazinyl)-2-nitro-dibenz(b,f)(1,4)oxazepine; 2-chloro-1′-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

TCAs are generally used in single oral doses up to the equivalent of 150 mg of imipramine. TCAs are metabolized via oxidation by hepatic microsomal enzymes followed by conjugation with glucuronic acid. TCA metabolites may be substituted for secondary amine tricyclic antidepressants, such as maprotiline, in the anti-inflammatory combination of the invention. The 10-hydroxy metabolites of TCAs are particularly useful in the methods of the invention, given that they have the biological activities of the original tricyclic antidepressant, but are less toxic.

TCA Standard Recommended Dosages

Typically, patient dosages of maprotiline vary according to the patient's condition, but some standard recommended dosages are provided herein. Maprotiline, which is currently available in 25, 50, and 100 mg tablets, is most often administered in doses of 100-150 mg/day, although standard recommended dosages of 1-25 mg/day, 25-100 mg/day, 100-150 mg/day, 150-225 mg/day, or 225-350 mg/day can be administered. Most antidepressants are well absorbed when administered orally, although intramuscular administration of some TCAs (e.g., amitriptyline, clomipramine) is also possible.

Triclosan

In one embodiment, the methods, compositions, and kits of the invention employ triclosan or another phenoxy phenol, or a structural or functional analog thereof in combination with a non-steroidal immunophilin-dependent immunosuppressant (NsIDI).

Triclosan is a chloro-substituted phenoxy phenol that acts as a broad-spectrum antibiotic. We report herein that triclosan also increases the potency of immunosuppressive agents, such as cyclosporine, and is useful in the anti-inflammatory combination of the invention for the treatment of an immunoinflammatory disorder, proliferative skin disease, organ transplant rejection, or graft versus host disease. Triclosan structural analogs include chloro-substituted phenoxy phenols, such as 5-chloro-2-(2,4-dichlorophenoxy)phenol, hexachlorophene, dichlorophene, as well as other halogenated hydroxydiphenyl ether compounds. Triclosan functional analogs include clotrimazole as well as various antimicrobials such as selenium sulfide, ketoconazole, triclocarbon, zinc pyrithione, itraconazole, asiatic acid, hinokitiol, mipirocin, clinacycin hydrochloride, benzoyl peroxide, benzyl peroxide, minocyclin, octopirox, ciclopirox, erythromycin, zinc, tetracycline, azelaic acid and its derivatives, phenoxy ethanol, ethylacetate, clindamycin, meclocycline. Functional and/or structural analogs of triclosan are also described, e.g., in U.S. Pat. Nos. 5,043,154, 5,800,803, 6,307,049, and 6,503,903.

Triclosan may achieve its anti-bacterial activity by binding to and inhibiting the bacterial enzyme Fab1, which is required for bacterial fatty acid synthesis. Triclosan structural or functional analogs, including antibiotics that bind Fab1, may also be useful in the combinations of the invention.

Triclosan Standard Recommended Dosages

While suggested dosages will vary with a patient's condition, standard recommended dosages are provided below. Typically, a patient will receive 3.24 mg per kg, although amounts between 0.5 and 3.24, or 3.24 and 5.0 may also be used. Other useful triclosan dosages include 0.5 mg/kg, 1.0 mg/kg, 1.5 mg/kg, 2.0 mg/kg, 2.5 mg/kg, 3.0 mg/kg, 3.5 mg/kg, 4.0 mg/kg, and 4.5 mg/kg for humans. Preferably, triclosan is applied topically in a formulation containing 0.5 to 3% triclosan. Other useful formulations contain 0.1%, 0.5%, 1%, 2%, 3%, 4%, 5%, 7.5%, or 10% triclosan.

Antihistamines

In yet another embodiment of the invention, the methods, compositions, and kits of the invention employ a histamine receptor antagonist (or analog thereof) and a non-steroidal immunophilin-dependent inhibitor to a patient in need of such treatment.

Antihistamines are compounds that block the action of histamine. Classes of antihistamines include:

(1) Ethanolamines (e.g., bromodiphenhydramine, carbinoxamine, clemastine, dimenhydrinate, diphenhydramine, diphenylpyraline, and doxylamine);

(2) Ethylenediamines (e.g., pheniramine, pyrilamine, tripelennamine, and triprolidine);

(3) Phenothiazines (e.g., diethazine, ethopropazine, methdilazine, promethazine, thiethylperazine, and trimeprazine);

(4) Alkylamines (e.g., acrivastine, brompheniramine, chlorpheniramine, desbrompheniramine, dexchlorpheniramine, pyrrobutamine, and triprolidine);

(5) piperazines (e.g., buclizine, cetirizine, chlorcyclizine, cyclizine, meclizine, hydroxyzine);

(6) Piperidines (e.g., astemizole, azatadine, cyproheptadine, desloratadine, fexofenadine, loratadine, ketotifen, olopatadine, phenindamine, and terfenadine);

(7) Atypical antihistamines (e.g., azelastine, levocabastine, methapyrilene, and phenyltoxamine).

In the methods, compositions, and kits of the invention, both non-sedating and sedating antihistamines may be employed. Particularly desirable antihistamines for use in the methods, compositions, and kits of the invention are non-sedating antihistamines such as loratadine and desloratadine. Sedating antihistamines can also be used in the methods, compositions, and kits of the invention. Preferred sedating antihistamines for use in the methods, compositions, and kits of the invention are azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the methods and compositions of the invention are acrivastine; ahistan; antazoline; astemizole; azelastine (e.g., azelsatine hydrochloride); bamipine; bepotastine; bietanautine; brompheniramine (e.g., brompheniramine maleate); carbinoxamine (e.g., carbinoxamine maleate); cetirizine (e.g., cetirizine hydrochloride); cetoxime; chlorocyclizine; chloropyramine; chlorothen; chlorphenoxamine; cinnarizine; clemastine (e.g., clemastine fumarate); clobenzepam; clobenztropine; clocinizine; cyclizine (e.g., cyclizine hydrochloride; cyclizine lactate); deptropine; dexchlorpheniramine; dexchlorpheniramine maleate; diphenylpyraline; doxepin; ebastine; embramine; emedastine (e.g., emedastine difumarate); epinastine; etymemazine hydrochloride; fexofenadine (e.g., fexofenadine hydrochloride); histapyrrodine; hydroxyzine (e.g., hydroxyzine hydrochloride; hydroxyzine pamoate); isopromethazine; isothipendyl; levocabastine (e.g., levocabastine hydrochloride); mebhydroline; mequitazine; methafurylene; methapyrilene; metron; mizolastine; olapatadine (e.g., olopatadine hydrochloride); orphenadrine; phenindamine (e.g., phenindamine tartrate); pheniramine; phenyltoloxamine; p-methyldiphenhydramine; pyrrobutamine; setastine; talastine; terfenadine; thenyldiamine; thiazinamium (e.g., thiazinamium methylsulfate); thonzylamine hydrochloride; tolpropamine; triprolidine; and tritoqualine.

Structural analogs of antihistamines may also be used in according to the invention. Antihistamine analogs include, without limitation, 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that are suitable for use in the invention are described in U.S. Pat. Nos. 3,956,296; 4,254,129; 4,254,130; 4,282,833; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

Antihistamine Standard Recommended Dosages

Standard recommended dosages for several exemplary antihistamines are shown in Table 3. Other standard dosages are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002).

TABLE 3

| Compound | Standard Dose |
|---|---|
| Desloratadine | 5 mg/once daily |
| Thiethylperazine | 10 mg/1-3 times daily |
| Bromodiphenhydramine | 12.5-25 mg/every 4-6 hours |
| Promethazine | 25 mg/twice daily |
| Cyproheptadine | 12-16 mg/day |
| Loratadine | 10 mg/once daily |
| Clemizole | 100 mg given as IV or IM |
| Azatadine | 1-2 mg/twice daily |
| Cetirizine | 5-10 mg/once daily |
| Chlorpheniramine | 2 mg/every 6 hours or |
| | 4 mg/every 6 hours |
| Dimenhydramine | 50-100 mg/every 4-6 hours |
| Diphenydramine | 25 mg/every 4-6 hours or |
| | 38 mg/every 4-6 hours* |
| Doxylamine | 25 mg/once daily or 12.5 mg/every |
| | four hours* |
| Fexofenadine | 60 mg/twice daily or 180 mg/once daily |
| Meclizine | 25-100 mg/day |
| Pyrilamine | 30 mg/every 6 hours |
| Tripelennamine | 25-50 mg/every 4 to 6 hours or 100 mg/twice |
| | daily (extended release)* |

An Exemplary Histamine Receptor Antagonist: Loratidine

Loratadine (CLARITIN) is a tricyclic piperidine that acts as a selective peripheral histamine H1-receptor antagonist. We report herein that loratadine and structural and functional analogs thereof, such as piperidines, tricyclic piperidines, histamine H1-receptor antagonists, are useful in the anti-immunoinflammatory combination of the invention for the treatment of immunoinflammatory disorders, transplanted organ rejection, and graft versus host disease.

Loratadine functional and/or structural analogs include other H1-receptor antagonists, such as AHR-11325, acrivastine, antazoline, astemizole, azatadine, azelastine, bromopheniramine, carebastine, cetirizine, chlorpheniramine, chlorcyclizine, clemastine, cyproheptadine, descarboethoxyloratadine, dexchlorpheniramine, dimenhydrinate, diphenylpyraline, diphenhydramine, ebastine, fexofenadine, hydroxyzine ketotifen, Iodoxamide, levocabastine, methdilazine, mequitazine, oxatomide, pheniramine pyrilamine, promethazine, pyrilamine, setastine, tazifylline, temelastine, terfenadine, trimeprazine, tripelennamine, triprolidine, utrizine, and similar compounds (described, e.g., in U.S. Pat. Nos. 3,956,296, 4,254,129, 4,254,130, 4,283,408, 4,362,736, 4,394,508, 4,285,957, 4,285,958, 4,440,933, 4,510,309, 4,550,116, 4,692,456, 4,742,175, 4,908,372, 5,204,249, 5,375,693, 5,578,610, 5,581,011, 5,589,487, 5,663,412, 5,994,549, 6,201,124, and 6,458,958).

Loratadine, cetirizine, and fexofenadine are second-generation H1-receptor antagonists that lack the sedating effects of many first generation H1-receptor antagonists. Piperidine H1-receptor antagonists include loratadine, cyproheptadine hydrochloride (PERIACTIN), and phenindiamine tartrate (NOLAHIST). Piperazine H1-receptor antagonists include hydroxyzine hydrochloride (ATARAX), hydroxyzine pamoate (VISTARIL), cyclizine hydrochloride (MAREZINE), cyclizine lactate, and meclizine hydrochloride.

Loratidine Standard Recommended Dosages

Loratadine oral formulations include tablets, redi-tabs, and syrup. Loratadine tablets contain 10 mg micronized loratadine. Loratadine syrup contains 1 mg/ml micronized loratadine, and reditabs (rapidly-disintegrating tablets) contain 10 mg micronized loratadine in tablets that disintegrate quickly in the mouth. While suggested dosages will vary with a patient's condition, standard recommended dosages are provided below. Loratadine is typically administered once daily in a 10 mg dose, although other daily dosages useful in the anti-immunoinflammatory combination of the invention include 0.01-0.05 mg, 0.05-1 mg, 1-3 mg, 3-5 mg, 5-10 mg, 10-15 mg, 15-20 mg, 20-30 mg, and 30-40 mg.

Loratadine is rapidly absorbed following oral administration. It is metabolized in the liver to descarboethoxyloratadine by cytochrome P450 3A4 and cytochrome P450 2D6. Loratadine metabolites are also useful in the anti-immunoinflammatory combination of the invention.

Phenothiazines

In another embodiment, the methods, compositions, and kits of the invention employ a phenothiazine, or a structural or functional analog thereof in combination with a non-steroidal immunophilin-dependent immunosuppressant (NsIDI).

Phenothiazines that are useful in the methods, compositions, and kits of the invention include compounds having the general formula (V):

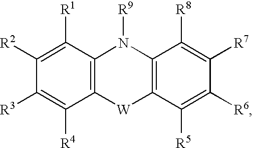

or a pharmaceutically acceptable salt thereof, wherein R² is selected from the group consisting of: CF₃, Cl, F, OCH₃, COCH₃, CN, OCF₃, COCH₂CH₃, CO(CH₂)₂CH₃, and SCH₂CH₃; R⁹ is selected from the group consisting of:

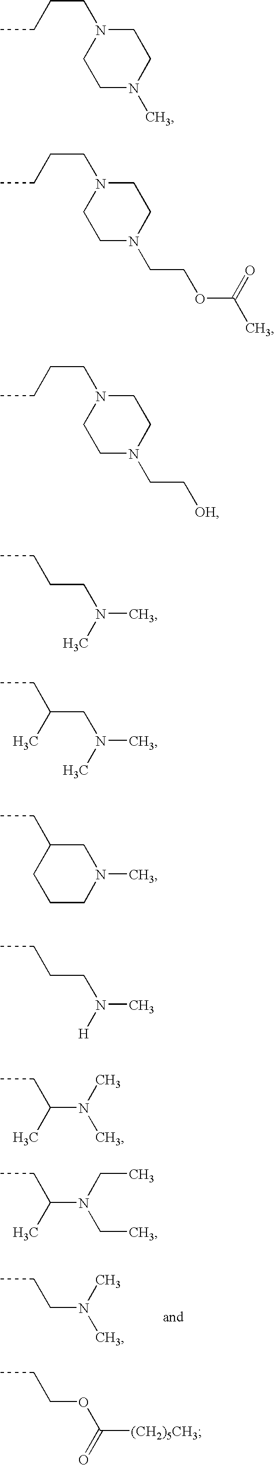

each of R¹, R³, R₄, R⁵, R₆, R⁷, and R⁸ is, independently, H, OH, F, OCF₃, or OCH₃; and W is selected from the group consisting of:

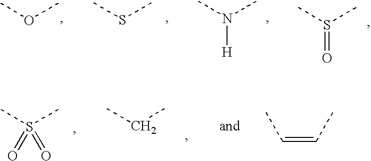

In some embodiments, the phenothiazine is a phenothiazine conjugate including a phenothiazine covalently attached via a linker to a bulky group of greater than 200 daltons or a charged group of less than 200 daltons. Such conjugates retain their anti-inflammatory activity in vivo and have reduced activity in the central nervous system in comparison to the parent phenothiazine. Phenothiazine conjugates that are useful in the methods, kits, and compositions of the invention are compounds having the general formula (VI).

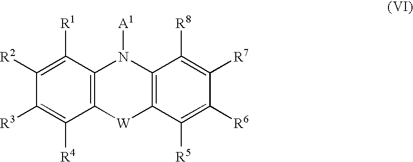

In formula (VI), R² is selected from the group consisting of: CF₃, halo, OCH₃, COCH₃, CN, OCF₃, COCH₂CH₃, CO(CH₂)₂CH₃, S(O)₂CH₃, S(O)₂N(CH₃)₂, and SCH₂CH₃; A¹ is selected from the group consisting of G¹,

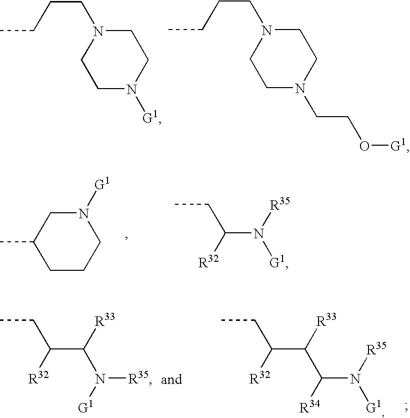

each of R¹, R³, R₄, R⁵, R₆, R⁷, and R⁸ is independently H, OH, F, OCF₃, or OCH₃; R³², R³³, R³⁴, and R³⁵, are each, independently, selected from H or C₁₋₆ alkyl; W is selected from the group consisting of: NO,

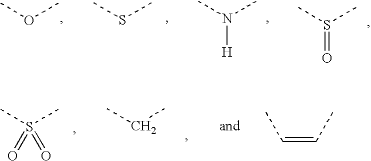

and G¹ is a bond between the phenothiazine and a linker, L.

The linker L is described by formula (VII):

G¹-(Z¹)ₒ-(Y¹)ᵤ-(Z²)ₛ-(R⁹)-(Z³)ₜ-(Y²)ᵥ-(Z⁴)ₚ-G² (VII)

In formula (VII), G¹ is a bond between the phenothiazine and the linker, G² is a bond between the linker and the bulky group or between the linker and the charged group, each of Z¹, Z², Z³, and Z⁴ is, independently, selected from O, S, and NR³⁹; R³⁹ is hydrogen or a C₁₋₆ alkyl group; each of Y¹ and Y² is, independently, selected from carbonyl, thiocarbonyl, sulphonyl, phosphoryl or similar acid-forming groups; o, p, s, t, u, and v are each independently 0 or 1; and R⁹ is a C₁₀ alkyl, a linear or branched heteroalkyl of 1 to 10 atoms, a C₂₋₀ alkene, a C₂₋₁₀ alkyne, a C₅₋₁₀ aryl, a cyclic system of 3 to 10 atoms, —(CH₂CH₂O)_{q}CH₂CH₂— in which q is an integer of 1 to 4, or a chemical bond linking G¹-(Z¹)ₒ-(Y¹)ᵤ-(Z²)ₛ- to -(Z³)ₜ-(Y²)_{v-(Z}⁴)ₚ-G².

The bulky group can be a naturally occurring polymer or a synthetic polymer. Natural polymers that can be used include, without limitation, glycoproteins, polypeptides, or polysaccharides. Desirably, when the bulky group includes a natural polymer, the natural polymer is selected from alpha-1-acid glycoprotein and hyaluronic acid. Synthetic polymers that can be used as bulky groups include, without limitation, polyethylene glycol, and the synthetic polypetide N-hxg.

The most commonly prescribed member of the phenothiazine family is chlorpromazine, which has the structure:

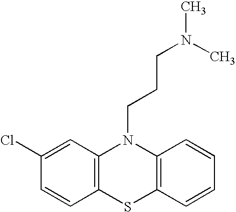

Chlorpromazine is a phenothiazine that has long been used to treat psychotic disorders. Phenothiazines include chlorpromazine functional and structural analogs, such as acepromazine, chlorfenethazine, chlorpromazine, cyamemazine, enanthate, fluphenazine, mepazine, mesoridazine besylate, methotrimeprazine, methoxypromazine, norchlorpromazine, perazine, perphenazine, prochlorperazine, promethazine, propiomazine, putaperazine, thiethylperazine, thiopropazate, thioridazine, trifluoperazine, or triflupromazine (or a salt of any of the above); and functional analogs that act as dopamine D2 antagonists (e.g., sulpride, pimozide, spiperone, clebopride, bupropion, and haloperidol).

Chlorpromazine is currently available in the following forms: tablets, capsules, suppositories, oral concentrates and syrups, and formulations for injection.

Because chlorpromazine undergoes extensive metabolic transformation into a number of metabolites that may be therapeutically active, these metabolites may be substituted for chlorpromazine in the anti-inflammatory combination of the invention. The metabolism of chlorpromazine yields, for example, oxidative N-demethylation to yield the corresponding primary and secondary amine, aromatic oxidation to yield a phenol, N-oxidation to yield the N-oxide, S-oxidation to yield the sulphoxide or sulphone, oxidative deamination of the aminopropyl side chain to yield the phenothiazine nuclei, and glucuronidation of the phenolic hydroxy groups and tertiary amino group to yield a quaternary ammonium glucuronide.

In other examples of chlorpromazine metabolites useful in the anti-inflammatory combination of the invention, each of positions 3, 7, and 8 of the phenothiazine can independently be substituted with a hydroxyl or methoxyl moiety.

Another phenothiazine is ethopropazine (brand name PARSITAN), an anticholinergic phenothiazine that is used as an antidyskinetic for the treatment of movement disorders, such as Parkinson's disease. Ethopropazine also has antihistaminic properties. We report herein that ethopropazine also increases the potency of immunosuppressive agents, such as cyclosporines. Unlike antipsychotic phenothiazines, which have three carbon atoms between position 10 of the central ring and the first amino nitrogen atom of the side chain at this position, strongly anticholinergic phenothiazines (e.g., ethopropazine, diethazine) have only two carbon atoms separating the amino group from position 10 of the central ring.

Ethopropazine structural analogs include trifluoroperazine dihydrochloride, thioridazine hydrochloride, and promethazine hydrochloride. Additional ethopropapazine structural analogs include 10-[2,3-bis(dimethylamino)propyl]phenothiazine, 10-[2,3-bis(dimethylamino)propyl]phenothiazine hydrochloride, 10-[2-(dimethylamino)propyl]phenothiazine; 10-[2-(dimethylamino)propyl]phenothiazine hydrochloride; and 10-[2-(diethylamino)ethyl]phenothiazine and mixtures thereof (see, e.g., U.S. Pat. No. 4,833,138).

Ethopropazine acts by inhibiting butyrylcholinesterase. Ethopropazine functional analogs include other anticholinergic compounds, such as Artane (trihexyphenidyl), Cogentin (benztropine), biperiden (U.S. Pat. No. 5,221,536), caramiphen, ethopropazine, procyclidine (Kemadrin), and trihexyphenidyl. Anticholinergic phenothiazines are extensively metabolized, primarily to N-dealkylated and hydroxylated metabolites. Ethopropazine metabolites may be substituted for ethopropazine in the anti-immunoinflammatory combination of the invention.

Phenothiazine Standard Recommended Dosages

Typically, patient dosage of chlorpromazine varies according to the patient's condition, but some standard recommended dosages are provided below. Chlorpromazine may be administered orally, by suppository, or by injection. Often doses are provided at intervals of 4-6 hours over the course of a day. Each dose is generally between 0.25-0.5 mg, 0.5-1.0 mg, 1-5 mg, 0.5-2 mg, 5-10 mg, 10-25 mg, 25-50 mg, 50-75 mg, or 75-100 mg. Generally, a total dose of 0.25 g, 0.50 g, 0.75 g, 1.0 g, 1.5 g, or 2.0 g is provided per day.

Ethopropazine, which is currently available in 10 and 50 mg tablets, is usually administered orally. Initially, patients are typically administered a 50 mg dose of ethopropazine once or twice a day. Other standard recommended dosages for ethopropazine are 1-10 mg/day, 10-25 mg/day, 50-100 mg/day, 100-400 mg/day, 500-600 mg/day, or 600-700 mg/day.

Mu Opioid Receptor Agonists

In yet another embodiment, the methods, compositions, and kits of the invention employ a mu opioid receptor agonist (or analog thereof) and a non-steroidal immunophilin-dependent inhibitor to a patient in need of such treatment. Loperamide hydrochloride (IMMODIUM) is a mu opioid receptor agonist useful in the treatment of diarrhea (U.S. Pat. No. 3,714,159). We report herein that loperamide and loperamide analogs increase the potency of an immunosuppressive agent and are useful in the treatment of an immunoinflammatory disorder, organ transplant rejection, or graft versus host disease. Loperamide is a piperidine butyramide derivative that is related to meperidine and diphenoxylate. It acts by relaxing smooth muscles and slowing intestinal motility. Other functionally and/or structurally related compounds, include meperidine, diphenoxylate, and related propanamines. Additional loperamide functional and structural analogs are described, e.g., in U.S. Pat. Nos. 4,066,654, 4,069,223, 4,072,686, 4,116,963, 4,125,531, 4,194,045, 4,824,853, 4,898,873, 5,143,938, 5,236,947, 5,242,944, 5,849,761, and 6,353,004. Loperamide functional analogs include peptide and small molecule mu opioid receptor agonists (described in U.S. Pat. No. 5,837,809). Such agents are also useful in the anti-inflammatory combination of the invention. Loperamide acts by binding to opioid receptors within the intestine and altering gastrointestinal motility.

Loperamide Standard Recommended Dosages

Loperamide is currently available in oral formulations as a 2 mg tablet. While suggested dosages will vary with a patient's condition, standard recommended dosages are provided below. Typically, an adult dose is 4 mg initially followed by subsequent 2 mg doses, or 16 mg per day. Other useful dosages include 0.5-1 mg, 1-2 mg, 2-4 mg, 4-8 mg, 8-12 mg, or 12-16 mg.

Corticosteroids

If desired, compositions and methods of the invention may be used with conventional therapeutics, including corticosteroids. One or more corticosteroid may be administered in a method of the invention or may be formulated with non-steroidal immunophilin-dependent enhancer, or analog or metabolite thereof, in a composition of the invention. Suitable corticosteroids include 11-alpha, 17-alpha, 21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha, 21-trihydroxy-6-alpha-methylpregn-4-ene-3,20-dione; 11-dehydrocorticosterone; 1′-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha, 20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxypredni solone; 9-fluorocorti sone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for various steroid/disease combinations are provided in Table 4, below.

TABLE 4
Standard Recommended Corticosteroid Dosages

| Indication | Route | Drug | Dose | | Schedule |
|---|---|---|---|---|---|
| Psoriasis | oral | prednisolone | 7.5-60 | mg | per day or divided b.i.d. |
| | oral | prednisone | 7.5-60 | mg | per day or divided b.i.d. |
| Asthma | inhaled | beclomethasone dipropionate | 42 | μg/puff) | 4-8 puffs b.i.d. |
| | inhaled | budesonide | (200 | μg/inhalation) | 1-2 inhalations b.i.d. |
| | inhaled | flunisolide | (250 | μg/puff) | 2-4 puffs b.i.d. |
| | inhaled | fluticasone propionate | (44, 110 or 220 | μg/puff) | 2-4 puffs b.i.d. |
| | inhaled | triamcinolone acetonide | (100 | μg/puff) | 2-4 puffs b.i.d. |
| COPD | oral | prednisone | 30-40 | mg | per day |
| Crohn's disease | oral | budesonide | 9 | mg | per day |
| Ulcerative colitis | oral | prednisone | 40-60 | mg | per day |
| | oral | hydrocortisone | 300 | mg (IV) | per day |
| | oral | methylprednisolone | 40-60 | mg | per day |
| Rheumatoid arthritis | oral | prednisone | 7.5-10 | mg | per day |

Other standard recommended dosages for corticbsteroids are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Steroid Receptor Modulators

Optionally, compositions and methods of the invention may be used in combination with steroid receptor modulators (e.g., antagonists and agonists) as a substitute for or in addition to a corticosteroid. Thus, in one embodiment, the invention features the combination of an NsIDI (or analog or metabolite thereof) and an NsIDIE and, optionally, a glucocorticoid receptor modulator or other steroid receptor modulator, and methods of treating immunoinflammatory disorders therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as in addition to a NsIDI/NsIDIE combination in the methods, compositions, and kits of the invention are A-348441 (Karo Bio), adrenal cortex extract (GlaxoSmithKline), alsactide (Aventis), amebucort (Schering A G), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering A G), fluocortolone monohydrate (Schering A G), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering A G), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering A G).

Therapy

The invention features methods for suppressing secretion of proinflammatory cytokines as a means for treating an immunoinflammatory disorder, proliferative skin disease, organ transplant rejection, or graft versus host disease. The suppression of cytokine secretion is achieved by administering one or more NsIDIEs in combination with one or more NsIDIs. While the examples describe particular NsIDIEs and NsIDIs, it is understood that a combination of multiple agents is often desirable. For example, methotrexate, hydroxychloroquine, and sulfasalazine are commonly administered for the treatment of rheumatoid arthritis. Additional therapies are described below.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to an NsIDI in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the invention features the combination of a tricyclic compound and a bronchodilator, and methods of treating COPD therewith.

Psoriasis

The methods, compositions, and kits of the invention may be used for the treatment of psoriasis. If desired, one or more antipsoriatic agents typically used to treat psoriasis may be used as a substitute for or in addition to an NsIDI in the methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of a tricyclic compound and an antipsoriatic agent, and methods of treating psoriasis therewith.

Inflammatory Bowel Disease

The methods, compositions, and kits of the invention may be used for the treatment of inflammatory bowel disease. If desired, one or more agents typically used to treat inflammatory bowel disease may be used as a substitute for or in addition to an NsIDI in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of a tricyclic compound and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used as a substitute for or in addition to an NsIDI in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of a tricyclic compound with any of the foregoing agents, and methods of treating rheumatoid arthritis therewith.

Asthma

The methods, compositions, and kits of the invention may be used for the treatment of asthma. If desired, one or more agents typically used to treat asthma may be used as a substitute for or in addition to an NsIDI in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of a tricyclic compound and any of the foregoing agents, and methods of treating asthma therewith.

Administration

In particular embodiments of any of the methods of the invention, an NsIDI and an NsIDIE are administered within 10 days of each other, within five days of each other, within twenty-four hours of each other, or simultaneously. The compounds may be formulated together as a single composition, or may be formulated and administered separately. One or both compounds may be administered in a low dosage or in a high dosage, each of which is defined herein. It may be desirable to administer to the patient other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the invention are especially useful for the treatment of immunoinflammatory disorders in combination with other anti-cytokine agents or agents that modulate the immune response to positively effect disease, such as agents that influence cell adhesion, or biologics (i.e., agents that block the action of IL-6, IL-1, IL-2, IL-12, IL-15 or TNF (e.g., etanercept, adelimumab, infliximab, or CDP-870). In this example (that of agents blocking the effect of TNFα), the combination therapy reduces the production of cytokines, etanercept or infliximab act on the remaining fraction of inflammatory cytokines, providing enhanced treatment.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Formulation of Pharmaceutical Compositions

The administration of a combination of the invention (e.g., an NsIDI/NsIDIE combination) may be by any suitable means that results in suppression of proinflammatory cytokine levels at the target region. A compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near simultaneous administration of the agents. Such co-formulated compositions can include the NsIDI and an NsIDIE formulated together in the same pill, capsule, liquid, etc. It is to be understood that, when referring to the formulation of “NsIDI/NsIDIE combinations,” the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Controlled Release Formulations

Administration of an NsIDI/NsIDIE combination of the invention in which one or both of the active agents is formulated for controlled release is useful where the NsIDI or the NsIDIE, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount of that is therapeutically effective for cytokine suppression. Accordingly, a sustained release formulation may be used to avoid frequent dosing that may be required in order to sustain the plasma levels of both agents at a therapeutic level. For example, in preferable oral pharmaceutical compositions of the invention, half-life and mean residency times from 10 to 20 hours for one or both agents of the combination of the invention are observed.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the NsIDI and/or the NsIDIE are released at period intervals, the release could be simultaneous, or a delayed release of one of the agents of the combination can be affected, when the early release of one particular agent is preferred over the other.

Controlled release formulations may include a degradable or nondegradable polymer, hydrogel, organogel, or other physical construct that modifies the bioabsorption, half-life or biodegradation of the agent. The controlled release formulation can be a material that is painted or otherwise applied onto the afflicted site, either internally or externally. In one example, the invention provides a biodegradable bolus or implant that is surgically inserted at or near a site of interest (for example, proximal to an arthritic joint). In another example, the controlled release formulation implant can be inserted into an organ, such as in the lower intestine for the treatment inflammatory bowel disease.

Hydrogels can be used in controlled release formulations for the NsIDI/NsIDIE combinations of the present invention. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylactic acid, polycaprolactone, polyorthoesters, polyanhydrides or polypeptides. The entire network can be gelled into a biodegradable network that can be used to entrap and homogeneously disperse NsIDI/NsIDIE combinations of the invention for delivery at a controlled rate.

Chitosan and mixtures of chitosan with carboxymethylcellulose sodium (CMC—Na) have been used as vehicles for the sustained release of drugs, as described by Inouye et al., Drug Design and Delivery 1: 297-305, 1987. Mixtures of these compounds and agents of the NsIDI/NsIDIE combinations of the invention, when compressed under 200 kg/cm², form a tablet from which the active agent is slowly released upon administration to a subject. The release profile can be changed by varying the ratios of chitosan, CMC—Na, and active agent(s). The tablets can also contain other additives, including lactose, CaHPO₄ dihydrate, sucrose, crystalline cellulose, or croscarmellose sodium. Several examples are given in Table 5.

TABLE 5

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄ * 2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose Na | | | | 110 | | | | | | | | |

Baichwal, in U.S. Pat. No. 6,245,356, describes a sustained release oral solid dosage forms that includes agglomerated particles of a therapeutically active medicament (for example, an NsIDI/NsIDIE combination or component thereof of the present invention) in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well-known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the combinations of the invention, Baichwal and Staniforth in U.S. Pat. No. 5,135,757 describe a free-flowing slow release granulation for use as a pharmaceutical excipient that includes from about 20 to about 70 percent or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and from about 30 to about 80 percent by weight of an inert pharmaceutical filler (such as, for example, lactose, dextrose, sucrose, sorbitol, xylitol, fructose or mixtures thereof). After mixing the excipient with an NsIDI/NsIDIE combination, or combination agent, of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the combinations of the invention, Shell, in U.S. Pat. No. 5,007,790, describe sustained-release oral drug-dosage forms that release a drug in solution at a rate controlled by the solubility of the drug. The dosage form comprises a tablet or capsule that includes a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles, assuring that drug reaches the stomach in the solution state which is less injurious to the stomach than solid-state drug. The programmed eventual dissolution of the polymer depends upon the nature of the polymer and the degree of crosslinking. The polymer is nonfibrillar and substantially water soluble in its uncrosslinked state, and the degree of crosslinking is sufficient to enable the polymer to remain insoluble for the desired time period, normally at least from about 4 hours to 8 hours up to 12 hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of the NsIDI/NsIDIE combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500 to 1000 μm size range.

Slow-release formulations can include a coating which is not readily water-soluble but which is slowly attacked and removed by water, or through which water can slowly permeate. Thus, for example, the NsIDI/NsIDIE combinations of the invention can be spray-coated with a solution of a binder under continuously fluidizing conditions, such as describe by Kitamori et al., U.S. Pat. No. 4,036,948. Examples of water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, organic solvent-soluble binders, such as cellulose derivatives (e.g., cellulose acetate phthalate, hydroxypropylmethyl-cellulose phthalate, ethylcellulose).

Combinations of the invention, or a component thereof, with sustained release properties can also be formulated by spray drying techniques. Yet another form of sustained release NsIDI/NsIDIE combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, for example, Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

Extended- and/or controlled-release formulations of NsIDIEs, such as SSRIs are known. For example, Paxil CR®, commercially available from GlaxoSmithKline, is an extended release form of paroxetine hydrochloride in a degradable polymeric matrix (GEOMATRIX™, see also U.S. Pat. Nos. 4,839,177, 5,102,666, and 5,422,123), which also has an enteric coat to delay the start of drug release until after the tablets have passed through the stomach. For example, U.S. Pat. No. 5,102,666 describes a polymeric controlled release composition comprising a reaction complex formed by the interaction of (1) a calcium polycarbophil component which is a water-swellable, but water insoluble, fibrous cross-linked carboxy-functional polymer, the polymer containing (a) a plurality of repeating units of which at least about 80% contain at least one carboxyl functionality, and (b) about 0.05 to about 1.5% cross-linking agent substantially free from polyalkenyl polyether, the percentages being based upon the weights of unpolymerised repeating unit and cross-linking agent, respectively, with (2) water, in the presence of an active agent selected from the group consisting of SSRIs such as paroxetine. The amount of calcium polycarbophil present is from about 0.1 to about 99% by weight, for example about 10%. The amount of active agent present is from about 0.0001 to about 65% by weight, for example between about 5 and 20%. The amount of water present is from about 5 to about 200% by weight, for example between about 5 and 10%. The interaction is carried out at a pH of between about 3 and about 10, for example about 6 to 7. The calcium polycarbophil is originally present in the form of a calcium salt containing from about 5 to about 25% calcium.

Other extended-release formulation examples are described in U.S. Pat. No. 5,422,123. Thus, a system for the controlled release of an active substance which is an SSRI such as paroxetine, comprising (a) a deposit-core comprising an effective amount of the active substance and having defined geometric form, and (b) a support-plafform applied to the deposit-core, wherein the deposit-core contains at least the active substance, and at least one member selected from the group consisting of (1) a polymeric material which swells on contact with water or aqueous liquids and a gellable polymeric material wherein the ratio of the swellable polymeric material to the gellable polymeric material is in the range 1:9 to 9:1, and (2) a single polymeric material having both swelling and gelling properties, and wherein the support-platform is an elastic support, applied to said deposit-core so that it partially covers the surface of the deposit-core and follows changes due to hydration of the deposit-core and is slowly soluble and/or slowly gellable in aqueous fluids. The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

In another example, an extended-release formulation for venlafaxine (Effexor XR®) is commercially available from Wyeth Pharmaceuticals. This formulation includes venlafaxine hydrochloride, microcrystalline cellulose and hydroxypropylmethylcellulose, coated with a mixture of ethyl cellulose and hydroxypropylmethylcellulose (see U.S. Pat. Nos. 6,403,120 and 6,419,958).

A controlled-release formulation of budesonide (3 mg capsules) for the treatment of inflammatory bowel disease is available from AstraZeneca (sold as “Entocort™”). To make low dose levels of active substance possible, the active substance is micronised, suitably mixed with known diluents, such as starch and lactose, and granulated with PVP (polyvinylpyrrolidone). Further, the granulate is laminated with a sustained release inner layer resistant to a pH of 6.8 and a sustained release outer layer resistant to a pH of 1.0. The inner layer is made of Eudragit®RL (copolymer of acrylic and methacrylic esters with a low content of quaternary ammonium groups) and the outer layer is made of Eudragit®L (anionic polymer synthesized from methacrylic acid and methacrylic acid methyl ester).

A bilayer tablet can be formulated for an NsIDI/NsIDIE combination of the invention in which different custom granulations are made for each agent of the combination and the two agents are compressed on a bi-layer press to form a single tablet. For example, 12.5 mg, 25 mg, 37.5 mg, or 50 mg of paroxetine, an NsIDIE, is formulated for a controlled release that results in a paroxetine t_{1/2} of 15 to 20 hours may be combined in the same tablet with cyclosporine, which is formulated such that the t_{1/2} approximates that of paroxetine. Examples of paroxetine extended-release formulations, including those used in bilayer tablets, can be found in U.S. Pat. No. 6,548,084. In addition to controlling the rate of cyclosporine release in vivo, an enteric or delayed release coat may be included that delays the start of drug release such that the Tₘₐₓ of cyclosporine approximates that of paroxetine (i.e. 5 to 10 hours).

Cyclodextrins are cyclic polysaccharides containing naturally occurring D(+)-glucopyranose units in an α-(1,4) linkage. Alpha-, beta- and gamma-cyclodextrins, which contain, respectively, six, seven or eight glucopyranose units, are most commonly used and suitable examples are described in WO91/11172, WO94/02518 and WO98/55148. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); Cserhati, Analytical Biochemistry 225: 328-32, 1995; Husain et al., Applied Spectroscopy 46: 652-8, 1992. Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules. U.S. Pat. No. 4,727,064 describes pharmaceutical preparations consisting of a drug with substantially low water solubility and an amorphous, water-soluble cyclodextrin-based mixture in which the drug forms an inclusion complex with the cyclodextrins of the mixture.

Formation of a drug-cyclodextrin complex can modify the drug's solubility, dissolution rate, bioavailability, and/or stability properties.

Sulfobutylether-β-cyclodextrin (SBE-β-CD, commercially available from CyDex, Inc, Overland Park, Kans., USA and sold as CAPTISOL®) can also be used as an aid in the preparation of sustained-release formulations of agents of the combinations of the present invention. For example, a sustained-release tablet has been prepared that includes prednisolone and SBE-β-CD compressed in a hydroxypropyl methylcellulose matrix (see Rao et al., J. Pharm. Sci. 90: 807-16, 2001). In another example of the use of various cyclodextrins, EP 1109806 B1 describes cyclodextrin complexes of paroxetine, where α-, β-, or γ-cyclodextrins, including eptakis(2-6-di-α-methyl)-β-cyclodextrin, (2,3,6-tri-O-methyl)-β-cyclodextrin, monosuccinyl eptakis(2,6-di-O-methyl)-β-cyclodextrin, or 2-hydroxypropyl-β-cyclodextrin] in anhydrous or hydrated form formed complex ratios of agent to cyclodextrin of from 1:0.25 to 1:20 can be obtained.

Polymeric cyclodextrins have also been prepared, as described in U.S. patent application Ser. Nos. 10/021,294 and 10/021,312. The cyclodextrin polymers so formed can be useful for formulating agents of the combinations of the present invention. These multifunctional polymeric cyclodextrins are commercially available from Insert Therapeutics, Inc., Pasadena, Calif., USA.

As an alternative to direct complexation with agents, cyclodextrins may be used as an auxiliary additive, e.g. as a carrier, diluent or solubiliser. Formulations that include cyclodextrins and other agents of the combinations of the present invention (i.e., an NsIDI or NsIDIE) can be prepared by methods similar to the preparations of the cyclodextrin formulations described herein.

Liposomal Formulations

One or both components of an NsIDI/NsIDIE combination of the invention, or mixtures of the two components together, can be incorporated into liposomal carriers for administration. The liposomal carriers are composed of three general types of vesicle-forming lipid components. The first includes vesicle-forming lipids which will form the bulk of the vesicle structure in the liposome. Generally, these vesicle-forming lipids include any amphipathic lipids having hydrophobic and polar head group moieties, and which (a) can form spontaneously into bilayer vesicles in water, as exemplified by phospholipids, or (b) are stably incorporated into lipid bilayers, with its hydrophobic moiety in contact with the interior, hydrophobic region of the bilayer membrane, and its polar head group moiety oriented toward the exterior, polar surface of the membrane.

The vesicle-forming lipids of this type are preferably ones having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. The above-described lipids and phospholipids whose acyl chains have a variety of degrees of saturation can be obtained commercially, or prepared according to published methods. Other lipids that can be included in the invention are glycolipids and sterols, such as cholesterol.

The second general component includes a vesicle-forming lipid which is derivatized with a polymer chain which will form the polymer layer in the composition. The vesicle-forming lipids which can be used as the second general vesicle-forming lipid component are any of those described for the first general vesicle-forming lipid component. Vesicle forming lipids with diacyl chains, such as phospholipids, are preferred. One exemplary phospholipid is phosphatidylethanolamine (PE), which provides a reactive amino group which is convenient for coupling to the activated polymers. An exemplary PE is distearyl PE (DSPE).

The preferred polymer in the derivatized lipid, is polyethyleneglycol (PEG), preferably a PEG chain having a molecular weight between 1,000-15,000 daltons, more preferably between 2,000 and 10,000 daltons, most preferably between 2,000 and 5,000 daltons. Other hydrophilic polymers which may be suitable include polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose.

Additionally, block copolymers or random copolymers of these polymers, particularly including PEG segments, may be suitable. Methods for preparing lipids derivatized with hydrophilic polymers, such as PEG, are well known e.g., as described in U.S. Pat. No. 5,013,556.

A third general vesicle-forming lipid component, which is optional, is a lipid anchor by which a targeting moiety is anchored to the liposome, through a polymer chain in the anchor. Additionally, the targeting group is positioned at the distal end of the polymer chain in such a way so that the biological activity of the targeting moiety is not lost. The lipid anchor has a hydrophobic moiety which serves to anchor the lipid in the outer layer of the liposome bilayer surface, a polar head group to which the interior end of the polymer is covalently attached, and a free (exterior) polymer end which is or can be activated for covalent coupling to the targeting moiety. Methods for preparing lipid anchor molecules of this types are described below.

The lipids components used in forming the liposomes are preferably present in a molar ratio of about 70-90 percent vesicle forming lipids, 1-25 percent polymer derivatized lipid, and 0.1-5 percent lipid anchor. One exemplary formulation includes 50-70 mole percent underivatized PE, 20-40 mole percent cholesterol, 0.1-1 mole percent of a PE-PEG (3500) polymer with a chemically reactive group at its free end for coupling to a targeting moiety, 5-10 mole percent PE derivatized with PEG 3500 polymer chains, and 1 mole percent alpha-tocopherol.

The liposomes are preferably prepared to have substantially homogeneous sizes in a selected size range, typically between about 0.03 to 0.5 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less.

The liposomal formulations of the present invention include at least one surface-active agent. Suitable surface-active agents useful for the formulation of the NsIDI/NsIDIE combinations described herein include compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants. Commercially available examples for each class of excipient are provided below.

Polyethoxylated fatty acids may be used as excipients for the formulation of NsIDI/NsIDIE combinations described herein. Examples of commercially available polyethoxylated fatty acid monoester surfactants include: PEG 4-100 monolaurate (Crodet L series, Croda), PEG 4-100 monooleate (Crodet 0 series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster K1, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapegg 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS # 9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet 040, Croda), PEG-40 stearate (Emerest® 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet 0-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of one or both components of an NsIDI/NsIDIE combinations according to the invention may include one or more of the polyethoxylated fatty acids above.

Polyethylene glycol fatty acid diesters may also be used as excipients for the NsIDI/NsIDIE combinations described herein. Examples of commercially available polyethylene glycol fatty acid diesters include: PEG-4 dilaurate (Mapeg® 200 DL, PPG), PEG-4 dioleate (Mapegg 200 DO, PPG), PEG-4 distearate (Kessco® 200 DS, Stepan), PEG-6 dilaurate (Kessco® PEG 300 DL, Stepan), PEG-6 dioleate (Kesscog PEG 300 DO, Stepan), PEG-6 distearate (Kesscog PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessc® PEG 1000 DS, Stepan), PEG-32 dilaurate (Kessco® PEG 1540 DL, Stepan), PEG-32 dioleate (Kesscog PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of an NsIDI/NsIDIE combination according to the invention may include one or more of the polyethylene glycol fatty acid diesters above.

PEG-fatty acid mono- and di-ester mixtures may be used as excipients for the formulation of an NsIDI/NsIDIE combination described herein. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of the NsIDI/NsIDIE combinations according to the invention may include one or more of the PEG-fatty acid mono- and di-ester mixtures above.

In addition, polyethylene glycol glycerol fatty acid esters may be used as excipients for the formulation of the NsIDI/NsIDIE combinations described herein. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® 0, Goldschmidt), and PEG-30 glyceryl oleate (Tagat® O₂, Goldschmidt). Formulations of the an NsIDI/NsIDIE combinations according to the invention may include one or more of the polyethylene glycol glycerol fatty acid esters above.

Alcohol-oil transesterification products may also be used as excipients for the formulation of the NsIDI/NsIDIE combinations described herein. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PR_{T} 56, Pulcra SA), PEG-60 castor oil (Nikkol CO-60TX, Nikko), PEG-100 castor oil, PEG-200 castor oil (Eumulging PR_{T} 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO— 10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labraface Hydro, Gattefosse), PEG-8 caprylic/capric glycerides (Labrasol, Gattefosse), PEG-6 caprylic/capric glycerides (SOFTIGEN® 767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol D S, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants. Formulations of the NsIDI/NsIDIE combinations according to the invention may include one or more of the alcohol-oil transesterification products above.

Polyglycerized fatty acids may also be used as excipients for the formulation of the NsIDI/NsIDIE combinations described herein. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor GO32, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G40, ABITEC), polyglyceryl-10 decaisostearate (Nikkol Decaglyn 10-IS, Nikko), polyglyceryl-101 decaoleate (Drewpol 10⁻¹⁰-O, Stepan), polyglyceryl-10 mono, dioleate (Caprol® PGE 860, ABITEC), and polyglyceryl polyricinoleate (Polymuls, Henkel). Formulations of the an NsIDI/NsIDIE combinations according to the invention may include one or more of the polyglycerized fatty acids above.

In addition, propylene glycol fatty acid esters may be used as excipients for the formulation of the an NsIDI/NsIDIE combinations described herein. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-06, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kesscog PGDS, Stepan), propylene glycol dicaprylate (Nikkol Sefsol 228, Nikko), and propylene glycol dicaprate (Nikkol PDD, Nikko). Formulations of the NsIDI/NsIDIE combinations of the invention may include one or more of the propylene glycol fatty acid esters above.

Mixtures of propylene glycol esters and glycerol esters may also be used as excipients for the formulation of the NsIDI/NsIDIE combinations described herein. One preferred mixture is composed of the oleic acid esters of propylene glycol and glycerol (Arlacel 186). Examples of these surfactants include: oleic (ATMOS 300, ARLACEL 186, ICI), and stearic (ATMOS 150). Formulations of the NsIDI/NsIDIE combinations according to the invention may include one or more of the mixtures of propylene glycol esters and glycerol esters above.

Further, mono- and diglycerides may be used as excipients for the formulation of the NsIDI/NsIDIE combinations described herein. Examples of commercially available mono- and diglycerides include: monopalmitolein (C16:1) (Larodan), monoelaidin (C18:1) (Larodan), monocaproin (C6) (Larodan), monocaprylin (Larodan), monocaprin (Larodan), monolaurin (Larodan), glyceryl monomyristate (C, 14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitorg 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitorg 988, Huls), caprylic/capric glycerides (Imwitor® 742, Huls), Mono-and diacetylated monoglycerides (Myvacetg 9-45, Eastman), glyceryl monostearate (Aldo® MS, Arlacel 129, ICI), lactic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of the NsIDI/NsIDIE combinations according to the invention may include one or more of the mono- and diglycerides above.

Sterol and sterol derivatives may also be used as excipients for the formulation of the NsIDI/NsIDIE combinations described herein. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-30 soyasterol (Nikkol BPS-30, Nikko). Formulations of the NsIDI/NsIDIE combinations according to the invention may include one or more of the sterol and sterol derivatives above.

Polyethylene glycol sorbitan fatty acid esters may also be used as excipients for the formulation of the NsIDI/NsIDIE combinations described herein. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tween® 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS106, Nikko), PEG-20 sorbitan tristearate (Tweeng 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween®. 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tweeng 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of the NsIDI/NsIDIE combinations according to the invention may include one or more of the polyethylene glycol sorbitan fatty acid esters above.

In addition, polyethylene glycol alkyl ethers may be used as excipients for the formulation of the NsIDI/NsIDIE combinations described herein. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of the NsIDI/NsIDIE combinations according to the invention may include one or more of the polyethylene glycol alkyl ethers above.

Sugar esters may also be used as excipients for the formulation of the NsIDI/NsIDIE combinations described herein. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of the NsIDI/NsIDIE combinations according to the invention may include one or more of the sugar esters above.

Polyethylene glycol alkyl phenols are also useful as excipients for the formulation of the NsIDI/INsIDIE combinations described herein. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of the NsIDI/NsIDIE combinations to the invention may include one or more of the polyethylene glycol alkyl phenols above.

Polyoxyethylene-polyoxypropylene block copolymers may also be used as excipients for the formulation of the NsIDI/NsIDIE combinations described herein. These surfactants are available under various trade names, including one or more of Synperonic PE series (ICI), Pluronic(g) series (BASF), Lutrol (BASF), Supronic, Monolan, Pluracare, and Plurodac. The generic term for these copolymers is “poloxamer” (CAS 9003-11-6). These polymers have the formula (X):

HO(C₂H₄O)ₐ(C₃H₆O)_{b}(C₂H₄O)ₐH (X)

where “a” and “b” denote the number of polyoxyethylene and polyoxypropylene units, respectively. These copolymers are available in molecular weights ranging from 1000 to 15000 daltons, and with ethylene oxide/propylene oxide ratios between 0.1 and 0.8 by weight. Formulations of the NsIDI/NsIDIE combinations according to the invention may include one or more of the polyoxyethylene-polyoxypropylene block copolymers above.

Polyoxyethylenes, such as PEG 300, PEG 400, and PEG 600, may be used as excipients for the formulation of the NsIDI/NsIDIE combinations described herein.

Sorbitan fatty acid esters may also be used as excipients for the formulation of the NsIDI/NsIDIE combinations described herein. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of the NsIDI/NsIDIE combinations according to the invention may include one or more of the sorbitan fatty acid esters above.

Esters of lower alcohols (C₂ to C₄) and fatty acids (C₈ to C₁₈) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of the NsIDI/NsIDIE combinations according to the invention may include one or more of the lower alcohol fatty acid esters above.

In addition, ionic surfactants may be used as excipients for the formulation of the NsIDI/NsIDIE combinations described herein. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of the NsIDI/NsIDIE combinations according to the invention may include one or more of the ionic surfactants above.

The excipients present in the formulations of the invention are present in amounts such that the carrier forms a clear, or opalescent, aqueous dispersion of the NsIDI, the NsIDIE, or the NsIDI/NsIDIE combination sequestered within the liposome. The relative amount of a surface-active excipient necessary for the preparation of liposomal or solid lipid nanoparticulate formulations is determined using known methodology. For example, liposomes may be prepared by a variety of techniques, such as those detailed in Szoka et al, 1980. Multilamellar vesicles (MLVs) can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

Other established liposomal formulation techniques can be applied as needed. For example, the use of liposomes to facilitate cellular uptake is described in U.S. Pat. Nos. 4,897,355 and 4,394,448.

Additional Applications

The compounds of the invention can be employed in immunomodulatory or mechanistic assays to determine whether other combinations, or single agents, are as effective as the combination in inhibiting secretion or production of proinflammatory cytokines or modulating immune response using assays generally known in the art, examples of which are described herein. For example, candidate compounds may be combined with an NsIDIE (or metabolite or analog therein) or a NsIDI and applied to stimulated PBMCs. After a suitable time, the cells are examined for cytokine secretion or production or other suitable immune response. The relative effects of the combinations versus each other, and versus the single agents are compared, and effective compounds and combinations are identified.

The combinations of the invention are also useful tools in elucidating mechanistic information about the biological pathways involved in inflammation. Such information can lead to the development of new combinations or single agents for inhibiting inflammation caused by proinflammatory cytokines. Methods known in the art to determine biological pathways can be used to determine the pathway, or network of pathways affected by contacting cells stimulated to produce proinflammatory cytokines with the compounds of the invention. Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

The following examples are to illustrate the invention. They are not meant to limit the invention in any way.

### EXAMPLE 1

### Assay for Proinflammatory Cytokine-Suppressing Activity

Compound dilution matrices were assayed for the suppression of IFNγ, IL-1β, IL-2, IL-4, IL-5, and TNFα, as described below.

IFNγ

A 100 μL suspension of diluted human white blood cells contained within each well of a polystyrene 384-well plate (NalgeNunc) was stimulated to secrete IFNγ by treatment with a final concentration of 10 ng/mL phorbol 12-myristate 13-acetate (Sigma, P-1585) and 750 ng/mL ionomycin (Sigma, I-0634). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-IFNγ antibody (Endogen, #M-700A-E). After a two-hour incubation, the plate was washed (Tecan PowerWasher 384) with phosphate buffered saline (PBS) containing 0.1% Tween 20 (polyoxyethylene sorbitan monolaurate) and incubated for an additional one hour with another anti-IFNγ antibody that was biotin labeled (Endogen, M701 B) and horseradish peroxidase (HRP) coupled to strepavidin (PharMingen, #13047E). After the plate was washed with 0.1% Tween 20/PBS, an HRP-luminescent substrate was added to each well and light intensity measured using a LJL Analyst plate luminometer.

IL-2

A 100 μL suspension of diluted human white blood cells contained within each well of a polystyrene 384-well plate (NalgeNunc) was stimulated to secrete IL-2 by treatment with a final concentration of 10 ng/mL phorbol 12-myristate 13-acetate (Sigma, P-1585) and 750 ng/mL ionomycin (Sigma, 1-0634). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-IL-2 antibody (PharMingen, #555051). After a two-hour incubation, the plate was washed (Tecan PowerWasher 384) with PBS containing 0.1% Tween 20 and incubated for an additional one hour with another anti-IL-2 antibody that was biotin labeled (Endogen, M600B) and HRP coupled to strepavidin (PharMingen, #13047E). After the plate was washed with 0.1% Tween 20/PBS, an HRP-luminescent substrate was added to each well and light intensity measured using a LJL Analyst plate luminometer.

TNFα Phorbol 12-Myistate 13-Acetate Stimulation

The effects of test compound combinations on TNFα secretion were assayed in white blood cells from human buffy coat stimulated with phorbol 12-myistate 13-acetate as follows. Human white blood cells from buffy coat were diluted 1:50 in media (RPMI; Gibco BRL, #11875-085), 10% fetal bovine serum (Gibco BRL, #25140-097), 2% penicillin/streptomycin (Gibco BRL, #15140-122)) and 50 μL of the diluted white blood cells was placed in each well of the assay plate. Drugs were added to the indicated concentration. After 16-18 hours of incubation at 37° C. with 5% CO₂ in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate was washed (Tecan Powerwasher 384) with PBS containing 0.1% Tween 20 and incubated for one additional hour with biotin labeled anti-TNFα antibody (PharMingen, #554511) and HRP coupled to streptavidin (PharMingen, #13047E). The plate was then washed again with 0.1% Tween 20/PBS. An HRP-luminescent substrate was added to each well, and the light intensity of each well was measured using a plate luminometer.

TNFα Lipopolysaccharide Stimulation

A 100 μl suspension of diluted human white blood cells contained within each well of a polystyrene 384-well plate (NalgeNunc) was stimulated to secrete TNFα by treatment with a final concentration of 2 μg/mL lipopolysaccharide (Sigma L-4130). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-TNFα antibody (PharMingen, #551220). After a two-hour incubation, the plate was washed (Tecan PowerWasher 384) with PBS containing 0.1% Tween 20 and incubated for an additional one hour with another anti-TNFα antibody that was biotin labeled (PharMingen, #554511) and HRP coupled to strepavidin (PharMingen, #13047E). After the plate was washed with 0.1% Tween 20/PBS, an HRP-luminescent substrate was added to each well and light intensity measured using a LJL Analyst plate luminometer.

Percent Inhibition

The percent inhibition (% I) for each well was calculated using the following formula:

% I=[(avg. untreated wells=treated well)/(avg. untreated wells)]×100

The average untreated well value (avg. untreated wells) is the arithmetic mean of 40 wells from the same assay plate treated with vehicle alone. Negative inhibition values result from local variations in treated wells as compared to untreated wells.

### EXAMPLE 2

### Preparation of Compounds

Stock solutions containing NsIDI and an NsIDIE were made in dimethylsulfoxide (DMSO) at a final concentration of between 0 and 40 μM. Master plates were prepared to contain dilutions of the stock solutions of the compounds described above. Master plates were sealed and stored at −20° C. until ready for use.

NsIDI Stocks

The stock solution containing cyclosporin A was made at a concentration of 1.2 mg/ml in DMSO. The stock solution of tacrolimus was made at a concentration of 0.04 mg/ml in DMSO.

NsIDIE Stocks

Stock solutions containing sertraline, fluoxetine, or fluvoxamine were made at a concentration of 10 mg/ml in DMSO. The stock solution containing maprotiline was made at a concentration of 10 mg/ml in DMSO. The stock solution containing triclosan was made at a concentration of 10 mg/mL in DMSO. The stock solution containing loratadine was made at a concentration of 10 mg/ml in DMSO. The stock solution containing chlorpromazine or ethopropazine was made at a concentration of 10 mg/mL in DMSO. The stock solution containing loperamide was made at a concentration of 10 mg/mL in DMSO. Master plates were prepared to contain dilutions of the stock solutions of the compounds described above. Master plates were sealed and stored at −20° C. until ready for use.

The final single agent plates were generated by transferring 1 μL of stock solution from the specific master plate to a dilution plate containing 100 μL of media (RPMI; Gibco BRL, #11875-085), 10% fetal bovine serum (Gibco BRL, #25140-097), 2% Penicillin/Streptomycin (Gibco BRL, # 15140-122)) using the Packard Mini-Trak liquid handler. This dilution plate was then mixed and a 5 μL aliquot transferred to the final assay plate, which had been pre-filled with 50 μL/well RPMI media containing the appropriate stimulant to activate IFNγ, IL-1β, IL-2, or TNFα secretion (see Example 1, supra).

### EXAMPLE 3

### The Combination of Cyclosporine A and Sertraline Reduces IL-2 Secretion In Vitro

IL-2 secretion was measured by ELISA as described above after stimulation with phorbol 12-myristate 13-acetate and ionomycin. The effects of varying concentrations of cyclosporine A, sertraline and a combination of sertraline and cyclosporine A were compared to control wells. These wells were stimulated with phorbol 12-myristate 13-acetate and ionomycin, but did not receive clyclosporine A or sertraline.

The results of this experiment are shown in Table 6. The effects of the agents alone and in combination are shown as percent inhibition of IL-2 secretion.

The data demonstrate that, in the present assay, cyclosporine A maximally inhibits IL-2 production by 83.5% at concentrations of 1 μM. The addition of 8 μM sertraline reduces the cyclosporine A concentration required for the same inhibition to 0.031 μM, a 32-fold reduction in the concentration of cyclosporine A.

TABLE 6
% Inhibition IL-2 PBMC PI

| | | Cyclosporine A (μM) | | | | | | | | |
|---|---|---|---|---|---|---|---|---|---|---|
| | | 0 | 0.008 | 0.016 | 0.031 | 0.062 | 0.125 | 0.25 | 0.5 | 1.0 |
| Sertraline (μM) | 0 | −0.4 | 0.0 | −1.7 | 18.6 | 44.4 | 68.5 | 75.1 | 80.6 | 83.5 |
| | 0.25 | 2.3 | 1.7 | 3.4 | 17.5 | 46.4 | 66.8 | 77.9 | 81.1 | 83.2 |
| | 0.5 | −2.9 | 0.6 | 13.1 | 22.2 | 48.5 | 71.4 | 79.5 | 82.6 | 84.2 |
| | 1 | 3.2 | −0.5 | 8.3 | 27.4 | 50.1 | 72.6 | 79.8 | 83.2 | 85.9 |
| | 2 | −0.8 | 9.0 | 6.4 | 28.5 | 64.4 | 79.1 | 83.8 | 87.0 | 87.4 |
| | 4 | 3.0 | 11.0 | 25.1 | 56.8 | 81.6 | 88.3 | 89.8 | 91.0 | 92.2 |
| | 8 | 20.8 | 34.9 | 55.7 | 85.4 | 92.4 | 94.5 | 95.2 | 95.5 | 95.4 |
| | 16 | 70.9 | 81.6 | 90.7 | 93.6 | 94.8 | 95.7 | 96.0 | 96.3 | 96.4 |
| | 32 | 86.3 | 90.1 | 89.2 | 92.2 | 90.1 | 95.7 | 96.2 | 95.8 | 91.5 |

### EXAMPLE 4

### The Combination of Cyclosporine A and Sertraline Reduces IFNγ Secretion In Vitro

IFNγ secretion was measured by ELISA as described above after stimulation with phorbol 12-myristate 13-acetate and ionomycin. The effect of varying concentrations of cyclosporine A, sertraline, and cyclosporine A in combination with sertraline was compared to control wells stimulated without cyclosporine A or sertraline. The results of this experiment are shown in Table 7, below. The effects of the agents alone and in combination are shown as percent inhibition of IFNγ secretion.

The data show that, in the present assay, cyclosporine A maximally inhibits IFNY production by 95.5% at concentrations of 1 μM. The addition of 8 μM sertraline demonstrates a dose sparing effect with cyclosporine A, nearly doubling the inhibition of IFNγ by 0.062 μM cyclosporine A, reaching 83.4% inhibition.

TABLE 7
% Inhibition IFN_{γ} PBMC PI

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 1.0 |
| Sertraline (μM) | 0 | −6.3 | 4.4 | 12.9 | 20.1 | 47.0 | 76.5 | 93.1 | 95.3 | 95.5 |
| | 0.25 | 0.0 | 5.6 | 8.6 | 18.6 | 41.8 | 78.1 | 93.2 | 95.3 | 95.4 |
| | 0.5 | 0.0 | −10.5 | 7.6 | 22.3 | 49.2 | 80.5 | 94.0 | 95.6 | 95.8 |
| | 1 | 4.5 | 5.7 | 11.4 | 22.9 | 47.4 | 82.3 | 93.9 | 95.4 | 95.7 |
| | 2 | 7.7 | 10.9 | 18.6 | 34.0 | 61.6 | 89.4 | 95.0 | 96.0 | 95.7 |
| | 4 | 26.0 | 29.0 | 33.5 | 46.3 | 71.4 | 91.2 | 95.7 | 96.7 | 96.8 |
| | 8 | 50.1 | 54.2 | 60.6 | 69.5 | 83.4 | 94.2 | 96.7 | 97.0 | 97.1 |
| | 16 | 78.2 | 82.8 | 80.9 | 85.2 | 91.9 | 96.0 | 97.3 | 97.6 | 96.6 |
| | 32 | 92.2 | 94.0 | 93.1 | 95.3 | 96.7 | 96.7 | 97.9 | 97.8 | 95.8 |

### EXAMPLE 5

### The Combination of Cyclosporine A and Sertraline Reduces TNFα Secretion In Vitro

TNFα secretion was measured by ELISA as described above after stimulation with phorbol 12-myristate 13-acetate and ionomycin. The effect of varying concentrations of cyclosporine A, sertraline, and cyclosporine A in combination with sertraline was compared to control wells stimulated without either cyclosporine A or sertraline. The results are shown in Table 8, below. The effects of the agents alone and in combination are shown as percent inhibition of TNFα secretion.

The data show that, in the present assay, cyclosporine A maximally inhibits TNFα production by 94.2% at concentrations of 1 μM. The addition of 8 μM sertraline demonstrates a dose sparing effect with cyclosporine A, doubling the inhibition of TNFα by 0.031 μM cyclosporine A, reaching 85.4% inhibition.

TABLE 8
% Inhibition TNFα PBMC PI

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 1.0 |
| Sertraline (μM) | 0 | −1.8 | 10.9 | 11.2 | 38.4 | 61.8 | 82.0 | 92.6 | 94.0 | 94.2 |
| | 0.25 | −1.8 | 10.6 | 14.0 | 32.0 | 60.5 | 81.1 | 92.7 | 94.1 | 93.3 |
| | .5 | −6.4 | 4.0 | 23.7 | 38.9 | 70.0 | 87.5 | 93.1 | 94.6 | 95.0 |
| | 1 | −0.4 | 13.2 | 22.7 | 40.9 | 63.9 | 88.7 | 92.3 | 95.3 | 95.4 |
| | 2 | −0.6 | 22.5 | 33.1 | 55.1 | 72.0 | 91.3 | 95.0 | 95.7 | 95.5 |
| | 4 | 23.5 | 37.8 | 46.8 | 62.0 | 84.6 | 94.6 | 95.9 | 96.4 | 96.9 |
| | 8 | 59.1 | 70.8 | 73.5 | 85.4 | 93.5 | 96.5 | 97.0 | 97.3 | 97.1 |
| | 16 | 73.8 | 93.4 | 92.4 | 95.7 | 97.4 | 97.6 | 98.2 | 95.0 | 97.7 |
| | 32 | 96.0 | 70.2 | 97.4 | 98.1 | 98.0 | 98.0 | 97.5 | 97.9 | 74.5 |

### EXAMPLE 6

### The Combination of Cyclosporine A and Fluoxetine Reduces IL-2 Secretion In Vitro

IL-2 secretion was measured by ELISA as described above after stimulation with phorbol 12-myristate 13-acetate and ionomycin. The effect of varying concentrations of cyclosporine A, fluoxetine, and cyclosporine A in combination with fluoxetine was compared to control wells stimulated without either cyclosporine A or fluoxetine. The results of this experiment are shown in Table 9, below. The effects of the agents alone and in combination are shown as percent inhibition of IL-2 secretion.

The data demonstrate that, in the present assay, that the addition of 21 μM fluoxetine in combination with 0.062 μM cyclosporine A inhibits IL-2 secretion by 98.8%, an enhancement of the inhibition 0.062 μM cyclosporine A provided alone.

TABLE 9
% Inhibition IL-2 PBMC PI

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 1.0 |
| Fluoxetine (μM) | 0 | −0.8 | 7.7 | 20.2 | 48.5 | 72.4 | 91.2 | 94.7 | 95.2 | 100.3 |
| | 0.65 | 0.8 | 12.7 | 15.8 | 47.3 | 75.1 | 86.7 | 92.9 | 94.6 | 98.4 |
| | 1.3 | −2.1 | 11.2 | 22.3 | 49.5 | 73.1 | 78.7 | 93.0 | 93.1 | 91.6 |
| | 2.6 | 0.6 | 8.8 | 28.3 | 47.2 | 71.3 | 84.7 | 91.5 | 93.1 | 92.2 |
| | 5.2 | −0.2 | 11.2 | 25.5 | 55.2 | 77.1 | 82.6 | 89.1 | 91.0 | 92.6 |
| | 10 | 16.1 | 24.3 | 45.5 | 66.5 | 91.2 | 91.3 | 93.6 | 92.4 | 89.4 |
| | 21 | 47.4 | 63.4 | 74.7 | 91.7 | 98.8 | 96.8 | 94.0 | 93.5 | 106.3 |
| | 42 | 90.3 | 94.2 | 91.7 | 105.2 | 109.8 | 109.3 | 102.0 | 107.0 | 106.0 |
| | 84 | 103.4 | 109.6 | 110.0 | 109.7 | 110.8 | 104.4 | 103.9 | 108.1 | 105.2 |

### EXAMPLE 7

### The Combination of Tacrolimus and Fluvoxamine Reduces IL-2 Secretion In Vitro

IL-2 secretion was measured by ELISA as described above after stimulation with phorbol 12-myristate 13-acetate and ionomycin. The effect of varying concentrations of tacrolimus, fluvoxamine, and tacrolimus in combination with fluvoxamine was compared to control wells stimulated without either tacrolimus or fluvoxamine. The results of this experiment are shown in Table 10, below. The effects of the agents alone and in combination are shown as percent inhibition of IL-2 secretion.

The data shows that, in the present assay, tacrolimus maximally inhibits IL-2 production by 87% at concentrations of 0.05 μM. The addition of 10 μM fluvoxamine demonstrates a dose sparing effect with cyclosporine A, reaching 85% inhibition of IL-2 with 0.013 μM tacrolimus.

TABLE 10
% Inhibition IL-2 PBMC PI

| | | Tacrolimus (μM) | | | | | | | | |
|---|---|---|---|---|---|---|---|---|---|---|
| | | 0 | 0.0004 | 0.0008 | 0.0016 | 0.0031 | 0.0062 | 0.013 | 0.025 | 0.05 |
| Fluvoxamine (μM) | 0 | −6.7 | 0.73 | −4.4 | 8.1 | 19 | 44 | 60 | 76 | 87 |
| | 0.16 | 1.1 | 2 | −1.1 | 13 | 17 | 39 | 63 | 79 | 86 |
| | 0.31 | 3.6 | 2.7 | 7.8 | 12 | 26 | 48 | 64 | 80 | 91 |
| | 0.62 | 4.6 | 1.7 | 7.4 | 8.8 | 17 | 43 | 62 | 80 | 90 |
| | 1.2 | −1.4 | −0.98 | 5.4 | 12 | 23 | 48 | 70 | 78 | 90 |
| | 2.5 | −2 | 7.9 | 2.9 | 7.1 | 30 | 55 | 68 | 83 | 91 |
| | 5 | 3.6 | 4.6 | 8 | 15 | 33 | 53 | 76 | 88 | 94 |
| | 10 | 8.1 | 14 | 10 | 25 | 48 | 70 | 85 | 92 | 97 |
| | 20 | 22 | 31 | 43 | 54 | 75 | 92 | 98 | 103 | 106 |

### EXAMPLE 8

### The Combination of Cyclosporine A and Paroxetine Reduces IL-2 Secretion In Vitro

IL-2 secretion was measured by ELISA as described above after stimulation with phorbol 12-myristate 13-acetate and ionomycin. The effect of varying concentrations of cyclosporine A, paroxetine, and cyclosporine A in combination with paroxetine was compared to control wells stimulated without cyclosporine A or paroxetine. The results of this experiment are shown in Table 11, below. The effects of the agents alone and in combination are shown as percent inhibition of IL-2 secretion.

The data show that, in the present assay, cyclosporine A inhibits IL-2 production by 97.7% at concentrations of 1 μM. The addition of 8.9 μM paroxetine demonstrates a dose sparing effect with cyclosporine A, reaching 90.7% inhibition of IL-2 with 0.062 μM cyclosporine A.

TABLE 11
% Inhibition IL-2 PBMC PI

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 1.0 |
| Paroxetine (μM) | 0 | 1.0 | −1.7 | 29.7 | 43.9 | 68.4 | 86.2 | 98.3 | 96.8 | 97.7 |
| | 0.56 | −2.4 | 5.0 | 23.4 | 47.6 | 69.1 | 85.1 | 91.5 | 97.9 | 102.7 |
| | 1.1 | −0.3 | 2.7 | 30.4 | 39.9 | 71.8 | 89.5 | 95.2 | 97.9 | 97.7 |
| | 2.2 | 4.8 | 10.5 | 26.8 | 42.7 | 69.6 | 88.5 | 95.4 | 92.1 | 100.4 |
| | 4.4 | 1.9 | 31.2 | 40.7 | 57.6 | 83.2 | 94.4 | 95.2 | 94.0 | 97.4 |
| | 8.9 | 21.6 | 38.7 | 61.3 | 74.1 | 90.7 | 91.9 | 92.5 | 95.9 | 92.2 |
| | 18 | 54.2 | 71.0 | 81.2 | 88.2 | 90.6 | 93.4 | 96.4 | 98.1 | 107.0 |
| | 36 | 83.5 | 89.8 | 94.3 | 102.5 | 100.5 | 99.5 | 99.1 | 104.3 | 100.7 |
| | 72 | 95.7 | 98.3 | 98.9 | 99.9 | 95.5 | 97.8 | 97.9 | 105.8 | 104.3 |

### EXAMPLE 9

### The Combination of Cyclosporine A and Paroxetine Reduces IL-2 Secrection In Vitro

IL-2 secretion was measured by ELISA as described above after stimulation with phorbol 12-myristate 13-acetate and ionomycin. The effect of varying concentrations of cyclosporine A, paroxetine, and cyclosporine A in combination with paroxetine was compared to control wells stimulated without cyclosporine A or paroxetine. The results of this experiment are shown in Table 12, below. The effects of the agents alone and in combination are shown as percent inhibition of IL-2 secretion.

TABLE 12
% Inhibition IL-2 PBMC PI

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 1.0 |
| Paroxetine (μM) | 0 | 1.0 | −1.7 | 29.7 | 43.9 | 68.4 | 86.2 | 98.3 | 96.8 | 97.7 |
| | 0.56 | −2.4 | 5.0 | 23.4 | 47.6 | 69.1 | 85.1 | 91.5 | 97.9 | 102.7 |
| | 1.1 | −0.3 | 2.7 | 30.4 | 39.9 | 71.8 | 89.5 | 95.2 | 97.9 | 97.7 |
| | 2.2 | 4.8 | 10.5 | 26.8 | 42.7 | 69.6 | 88.5 | 95.4 | 92.1 | 100.4 |
| | 4.4 | 1.9 | 31.2 | 40.7 | 57.6 | 83.2 | 94.4 | 95.2 | 94.0 | 97.4 |
| | 8.9 | 21.6 | 38.7 | 61.3 | 74.1 | 90.7 | 91.9 | 92.5 | 95.9 | 92.2 |
| | 18 | 54.2 | 71.0 | 81.2 | 88.2 | 90.6 | 93.4 | 96.4 | 98.1 | 107.0 |
| | 36 | 83.5 | 89.8 | 94.3 | 102.5 | 100.5 | 99.5 | 99.1 | 104.3 | 100.7 |
| | 72 | 95.7 | 98.3 | 98.9 | 99.9 | 95.5 | 97.8 | 97.9 | 105.8 | 104.3 |

### EXAMPLE 10

### The Combination of Cyclosporine A and Maprotiline Reduces IL-2 Secretion in vitro

IL-2 secretion was measured by ELISA as described above after stimulation with phorbol 12-myristate 13-acetate and ionomycin. The effects of varying concentrations of cyclosporine A, maprotiline, and a combination of maprotiline and cyclosporine A were compared to control wells. These wells were stimulated with phorbol 12-myristate 13-acetate and ionomycin, but did not receive cyclosporine A or maprotiline.

The results of this experiment are shown in Table 13. The effects of the agents alone and in combination are shown as percent inhibition of IL-2 secretion. These results were averaged from experiments carried out with white blood cells taken from two different donors.

TABLE 13
% Inhibition

| | | Cyclosporine A (μM) | | | | | | | | |
|---|---|---|---|---|---|---|---|---|---|---|
| | | 0.00 | 0.0032 | 0.0064 | 0.013 | 0.026 | 0.052 | 0.10 | 0.21 | 0.41 |
| Maprotiline (μM) | 0.00 | −15.60 | −12.75 | −13.52 | −8.52 | 11.51 | 34.60 | 63.75 | 77.15 | 81.65 |
| | 0.25 | −11.33 | −17.35 | −16.60 | −11.45 | 3.38 | 35.40 | 63.50 | 77.50 | 81.95 |
| | 0.50 | −13.60 | −11.69 | −13.59 | −9.68 | 3.42 | 41.85 | 74.55 | 75.35 | 81.10 |
| | 1.00 | −12.50 | −10.55 | −11.86 | −3.55 | 14.10 | 44.55 | 75.50 | 76.40 | 81.35 |
| | 2.00 | −11.75 | −12.52 | −6.86 | 5.82 | 20.83 | 59.30 | 76.45 | 77.70 | 80.00 |
| | 4.00 | 2.26 | 12.16 | 8.33 | 12.76 | 44.55 | 69.35 | 74.90 | 79.85 | 81.80 |
| | 8.00 | 42.00 | 43.50 | 46.70 | 53.50 | 69.95 | 77.75 | 84.30 | 84.85 | 86.15 |
| | 16.00 | 68.00 | 71.10 | 78.05 | 79.25 | 84.65 | 81.80 | 84.30 | 87.20 | 86.85 |
| | 32.00 | 77.90 | 81.60 | 83.25 | 81.65 | 85.00 | 85.95 | 84.65 | 86.75 | 86.15 |

### EXAMPLE 11

### The Combination of Cyclosporine A and Maprotiline Reduces TNFα Secretion In Vitro

TNFα secretion was measured by ELISA, as described above, following stimulation with lipopolysaccharide. The effect of varying concentrations of cyclosporine A, maprotiline, and cyclosporine A in combination with maprotiline was compared to control wells stimulated without cyclosporine A or maprotiline. The results are shown in Table 14. The effects of the agents alone and in combination are shown as percent inhibition of TNFα secretion. These results are the average of experiments carried out with white blood cells obtained from two donors.

TABLE 14
% Inhibition

| | | Cyclosporine A (μM) | | | | | | | | |
|---|---|---|---|---|---|---|---|---|---|---|
| | | 0.00 | 0.077 | 0.015 | 0.031 | 0.062 | 0.12 | 0.25 | 0.50 | 0.99 |
| Maprotiline (μM) | 0.00 | −3.84 | 19.67 | 35.90 | 54.90 | 84.05 | 92.80 | 95.80 | 94.60 | 95.75 |
| | 0.27 | −10.03 | 29.37 | 40.35 | 61.90 | 80.55 | 92.25 | 95.45 | 95.70 | 97.25 |
| | 0.54 | −9.41 | 21.82 | 40.25 | 60.25 | 77.90 | 92.95 | 97.90 | 96.60 | 96.15 |
| | 1.10 | −7.35 | 11.86 | 54.70 | 62.80 | 80.30 | 91.95 | 97.45 | 95.90 | 95.95 |
| | 2.20 | −3.53 | 7.69 | 57.20 | 65.00 | 85.60 | 94.00 | 94.75 | 97.40 | 95.95 |
| | 4.30 | 6.62 | 12.46 | 50.85 | 71.50 | 83.20 | 94.75 | 96.10 | 95.10 | 95.60 |
| | 8.60 | 8.37 | 30.85 | 57.80 | 71.05 | 87.85 | 94.70 | 95.75 | 97.10 | 96.50 |
| | 17.00 | 33.90 | 50.80 | 73.10 | 87.15 | 90.80 | 96.10 | 96.40 | 97.00 | 97.55 |
| | 35.00 | 70.25 | 90.65 | 92.25 | 96.00 | 97.15 | 94.85 | 96.45 | 97.70 | 97.95 |

### EXAMPLE 12

### The Combination of Cyclosporine A and Triclosan Reduces IL-2 Secretion In Vitro

IL-2 secretion was measured by ELISA as described above after stimulation with phorbol 12-myristate 13-acetate and ionomycin. The effects of varying concentrations of cyclosporine A, triclosan, and a combination of triclosan and cyclosporine A were compared to control wells. These wells were stimulated with phorbol 12-myristate 13-acetate and ionomycin, but did not receive cyclosporine A or triclosan.

The results of this experiment are shown in Table 15. The effects of the agents alone and in combination are shown as percent inhibition of IL-2 secretion. These results were averaged from experiments carried out with white blood cells taken from two different donors.

TABLE 15
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Triclosan (μM) | 0 | −7.8 | −1.2 | 22.3 | 39.6 | 62.6 | 86.1 | 94.1 | 94.5 | 95.9 |
| | 0.27 | −8.1 | −3.2 | 16.6 | 35.9 | 62.4 | 85.4 | 93.5 | 95.1 | 96.1 |
| | 0.54 | −4.7 | 0.7 | 17.4 | 40.3 | 62.7 | 88.6 | 94.1 | 96.0 | 96.8 |
| | 1.1 | 4.2 | 6.1 | 21.8 | 36.2 | 71.8 | 84.6 | 94.9 | 96.3 | 96.2 |
| | 2.2 | 1.2 | 8.1 | 14.8 | 33.2 | 71.4 | 89.4 | 94.7 | 95.9 | 95.6 |
| | 4.3 | 1.7 | 9.4 | 17.1 | 35.2 | 71.0 | 92.3 | 94.0 | 95.5 | 95.6 |
| | 8.6 | 1.7 | 11.9 | 24.6 | 53.7 | 78.1 | 91.6 | 95.1 | 95.2 | 96.6 |
| | 17 | 0.5 | 7.7 | 29.4 | 63.3 | 83.1 | 94.8 | 95.9 | 96.1 | 96.4 |
| | 35 | 53.8 | 82.5 | 86.1 | 94.2 | 96.6 | 97.4 | 96.7 | 97.8 | 97.4 |

### EXAMPLE 3

### The Combination of Cyclosporine A and Triclosan Reduces TNFαSecretion In Vitro

TNFα secretion was measured by ELISA as described above after stimulation with lipopolysaccharide. The effect of varying concentrations of cyclosporine A, triclosan, and cyclosporine A in combination with triclosan was compared to control wells stimulated without cyclosporine A or triclosan. The results of this experiment are shown in Table 16. The effects of the agents alone and in combination are shown as percent inhibition of TNFα secretion. The results of this experiment are the average of experiments carried out with white blood cells obtained from two donors.

TABLE 16
% Inhibition

| | | Cyclosporine (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Triclosan (μM) | 0 | −3.8 | 19.7 | 35.9 | 54.9 | 84.1 | 92.8 | 95.8 | 94.6 | 95.8 |
| | 0.27 | −10.0 | 29.4 | 40.4 | 61.9 | 80.6 | 92.3 | 95.5 | 95.7 | 97.3 |
| | 0.54 | −9.4 | 21.8 | 40.3 | 60.3 | 77.9 | 93.0 | 97.9 | 96.6 | 96.2 |
| | 1.1 | −7.3 | 11.9 | 54.7 | 62.8 | 80.3 | 92.0 | 97.5 | 95.9 | 96.0 |
| | 2.2 | −3.5 | 7.7 | 57.2 | 65.0 | 85.6 | 94.0 | 94.8 | 97.4 | 96.0 |
| | 4.3 | 6.6 | 12.5 | 50.9 | 71.5 | 83.2 | 94.8 | 96.1 | 95.1 | 95.6 |
| | 8.6 | 8.4 | 30.9 | 57.8 | 71.1 | 87.9 | 94.7 | 95.8 | 97.1 | 96.5 |
| | 17 | 33.9 | 50.8 | 73.1 | 87.2 | 90.8 | 96.1 | 96.4 | 97.0 | 97.6 |
| | 35 | 70.3 | 90.7 | 92.3 | 96.0 | 97.2 | 94.9 | 96.5 | 97.7 | 98.0 |

### EXAMPLE 14

### The Combination of Cyclosporine A and Loratadine Reduces IL-2 Secretion In Vitro

IL-2 secretion was measured by ELISA as described above after stimulation with phorbol 12-myristate 13-acetate and ionomycin. The effects of varying concentrations of cyclosporine A, loratadine, and a combination of loratadine and cyclosporine A were compared to control wells. These wells were stimulated with phorbol 12-myristate 13-acetate and ionomycin, but did not receive cyclosporine A or loratadine.

The results of this experiment are shown in Table 17. The effects of the agents alone and in combination are shown as percent inhibition of IL-2 secretion. The results shown below in are from a single representative experiment.

TABLE 17
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Loratadine (μM) | 0 | −20.0 | −8.2 | −7.7 | 13.3 | 46.1 | 77.9 | 86.6 | 93.1 | 92.8 |
| | 0.53 | −20.0 | −12.1 | −15.5 | 8.8 | 51.9 | 81.8 | 88.3 | 91.5 | 92.9 |
| | 1.1 | −17.8 | −18.3 | −20.0 | 7.2 | 50.2 | 81.2 | 78.9 | 92.3 | 93.6 |
| | 2.1 | −16.7 | −12.7 | −8.4 | 0.8 | 38.5 | 80.6 | 83.7 | 89.8 | 93.2 |
| | 4.3 | −20.0 | −20.0 | −8.4 | 9.9 | 52.6 | 79.4 | 87.8 | 91.1 | 91.8 |
| | 8.5 | −20.0 | −11.4 | −7.3 | 4.5 | 58.4 | 82.5 | 87.0 | 90.5 | 93.3 |
| | 17 | −20.0 | −16.1 | 2.8 | 22.8 | 70.6 | 84.6 | 88.6 | 92.9 | 93.6 |
| | 34 | −19.1 | −6.0 | 10.0 | 40.5 | 76.3 | 86.7 | 91.2 | 93.8 | 95.2 |
| | 68 | −4.3 | 7.5 | 22.3 | 70.1 | 87.8 | 92.4 | 95.0 | 95.4 | 95.9 |

### EXAMPLE 15

### The Combination of Cyclosporine A and Loratadine Reduces TNFα Secretion In Vitro

TNFα secretion was measured by ELISA as described above after stimulation with lipopolysaccharide. The effect of varying concentrations of cyclosporine A, loratadine, and cyclosporine A in combination with loratadine was comparted to control wells stimulated without cyclosporine A or loratadine. The results of this experiment are shown in Table 18 below. The effects of the agents alone and in combination are shown as percent inhibition of TNFα secretion. These results are the average of experiments carried out with white blood cells obtained from two donors. The results shown below are from a single representative experiment.

TABLE 18
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Loratadine (μM) | 0 | 10.4 | 10.4 | 24.7 | 63.0 | 82.5 | 90.2 | 89.4 | 84.0 | 87.7 |
| | 0.53 | 4.9 | 21.3 | 37.7 | 58.5 | 85.8 | 90.1 | 82.6 | 85.9 | 92.7 |
| | 1.1 | −3.8 | 33.0 | 39.6 | 54.7 | 84.4 | 89.4 | 91.5 | 92.1 | 92.4 |
| | 2.1 | 18.3 | 28.4 | 28.7 | 56.4 | 79.9 | 91.1 | 92.7 | 90.7 | 93.3 |
| | 4.3 | 9.2 | 26.2 | 32.9 | 55.1 | 84.9 | 90.4 | 93.3 | 93.0 | 94.2 |
| | 8.5 | 12.5 | 37.8 | 51.4 | 72.3 | 88.7 | 93.7 | 93.8 | 93.3 | 93.5 |
| | 17 | 42.1 | 48.9 | 62.1 | 80.4 | 90.2 | 97.1 | 94.1 | 95.6 | 95.1 |
| | 34 | 44.9 | 65.5 | 72.8 | 88.0 | 91.1 | 93.3 | 94.4 | 95.4 | 95.3 |
| | 68 | 69.8 | 73.5 | 89.0 | 87.5 | 95.9 | 97.1 | 93.3 | 96.7 | 96.8 |

### EXAMPLE 16

### The Combination of Cyclosporine A and Desloratadine Reduces TNFα Secretion In Vitro

TNFα secretion was assayed as described above after stimulation with phorbol 12-myristate 13-acetate. The effect of varying concentrations of cyclosporine and desloratidine was compared to control wells stimulated without cyclosporine A or loratadine. The results of this experiment are shown below in Table 19.

TABLE 19
% Inhibition

| | | CYCLOSPORINE (μM) | | | | | | | | |
|---|---|---|---|---|---|---|---|---|---|---|
| | | 0 | 0.0019 | 0.0039 | 0.0077 | 0.015 | 0.031 | 0.062 | 0.12 | 0.25 |
| DESLORATIDINE (μM) | 0 | −0.1777 | 1.953 | 0.975 | 6.922 | 17.44 | 33.95 | 55.98 | 72.58 | 90.68 |
| | 0.25 | −1.255 | 5.065 | 3.345 | 10.4 | 21.28 | 36.2 | 55.17 | 75.58 | 91.6 |
| | 0.51 | −4.652 | 3.805 | 5.8 | 5.505 | 14.89 | 32.55 | 58.65 | 79.03 | 92 |
| | 1 | 6.598 | 7.185 | 7.982 | 12.26 | 21.1 | 38.65 | 65.02 | 82.45 | 92.93 |
| | 2 | 10.61 | 15.79 | 19.43 | 25.43 | 32.85 | 51.05 | 66.6 | 84.27 | 92.53 |
| | 4.1 | 31.45 | 38.38 | 33 | 38.95 | 48.93 | 64.78 | 78.58 | 90.38 | 93.78 |
| | 8.1 | 56 | 58.73 | 60.02 | 63 | 71.58 | 78.9 | 87.2 | 93.77 | 95.15 |
| | 16 | 82.18 | 84.38 | 83.05 | 85.28 | 89.5 | 91.95 | 94.2 | 96 | 95.83 |
| | 33 | 89.4 | 95.05 | 94.75 | 94.97 | 96.07 | 95.45 | 94.42 | 96.8 | 95.62 |

### EXAMPLE 17

### Combination of Cyclosporine A and Loratidine Reduces TNFα Secretion In Vitro.

TNFα secretion was assayed as described above after stimulation with phorbol 12-myistate 13-acetate. The effect of varying concentrations of cyclosporine and loratidine was compared to control wells stimulated without cyclosporine A or loratadine. The results of this experiment are shown below in Table 20.

TABLE 20

| | | CYCLOSPORINE (μM) | | | | | | | | |
|---|---|---|---|---|---|---|---|---|---|---|
| | | 0 | 0.0019 | 0.0039 | 0.0077 | 0.015 | 0.031 | 0.062 | 0.12 | 0.25 |
| LORATADINE (μM) | 0 | −0.3725 | 1.825 | 5.875 | 11.71 | 25.85 | 52.45 | 75.95 | 89 | 91.95 |
| | 0.2 | 0 | 1.041 | 4.4 | 13.2 | 29.1 | 52.4 | 78.75 | 90.35 | 92.95 |
| | 0.41 | −2.384 | 2.075 | 3.525 | 11.39 | 27.15 | 49.7 | 79.05 | 90.55 | 91.15 |
| | 0.82 | 0.3615 | 0.16 | 8.96 | 13.9 | 31.4 | 53.5 | 81.75 | 91.3 | 91.65 |
| | 1.6 | 3.4 | 5.35 | 13.2 | 19.4 | 36.3 | 61.85 | 83.45 | 91.35 | 90.55 |
| | 3.3 | 4.83 | 14.5 | 5.785 | 24.7 | 38.2 | 63.5 | 84.5 | 89.25 | 91.15 |
| | 6.5 | 19.45 | 27.3 | 22.2 | 37.1 | 50.85 | 70.4 | 84.35 | 90.15 | 91 |
| | 13 | 30.1 | 36.95 | 36.15 | 46 | 61.45 | 73.9 | 88.1 | 91.65 | 92.7 |
| | 26 | 40.7 | 51.25 | 50.9 | 55.35 | 65.6 | 74.4 | 89.3 | 92.05 | 92.15 |

### EXAMPLE 18

### The Combination of Cyclosporine A and Chlorpromazine Reduces IL-2 Secretion In Vitro

IL-2 secretion was measured by ELISA as described above after stimulation with phorbol 12-myristate 13-acetate and ionomycin. The effects of varying concentrations of cyclosporine A, chlorpromazine, and a combination of chlorpromazine and cyclosporine A were compared to control wells. These wells were stimulated with phorbol 12-myristate 13-acetate and ionomycin, but did not receive cyclosporine A or chlorpromazine.

The results of this experiment are shown in Table 21. The effects of the agents alone and in combination are shown as percent inhibition of IL-2 secretion. The results shown below are from a single representative experiment.

TABLE 21
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Chlorpromazine (μM) | 0 | −14.1 | −11.7 | 0.35 | 28.8 | 55.6 | 74.0 | 78.6 | 80.1 | 82.3 |
| | 0.6 | −13.3 | −11.1 | −4.7 | 33.6 | 54.8 | 67.2 | 78.7 | 84.9 | 84.2 |
| | 1.2 | −18.7 | −10.8 | 4.6 | 28.0 | 57.8 | 73.4 | 78.0 | 81.9 | 83.2 |
| | 2.5 | −12.7 | −14.8 | −8.7 | 25.0 | 55.6 | 76.1 | 81.2 | 82.1 | 85.8 |
| | 5.0 | −13.7 | −5.9 | 6.7 | 36.1 | 66.1 | 77.4 | 81.3 | 85.7 | 86.8 |
| | 9.9 | −1.9 | 9.5 | 25.9 | 58.8 | 76.7 | 85.0 | 87.9 | 88.4 | 88.1 |
| | 20.0 | 24.7 | 49.6 | 67.4 | 84.0 | 89.2 | 92.0 | 91.5 | 93.3 | 89.8 |
| | 40.0 | 80.7 | 86.9 | 89.4 | 94.4 | 94.8 | 94.8 | 95.3 | 94.7 | 94.3 |
| | 80.0 | 94.70 | 92.1 | 94.9 | 89.3 | 95.8 | 92.7 | 93.3 | 94.9 | 94.3 |

### EXAMPLE 19

### The Combination of Cyclosporine A and Chlorpromazine Reduces TNFα Secretion In Vitro

TNFα secretion was measured by ELISA as described above after stimulation with lipopolysaccharide. The effect of varying concentrations of cyclosporine A, chlorpromazine, and cyclosporine A in combination with chlorpromazine was compared to control wells stimulated without cyclosporine A or chlorpromazine. The results of this experiment are shown in Table 22 below. The effects of the agents alone and in combination are shown as percent inhibition of TNFα secretion. These results are the average of experiments carried out with white blood cells obtained from two donors.

TABLE 22
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Chlorpromazine (μM) | 0.00 | −0.4 | 18.1 | 30.6 | 47.9 | 69.2 | 82.0 | 93.9 | 94.8 | 95.4 |
| | 0.27 | 4.9 | 28.0 | 37.9 | 54.0 | 73.6 | 88.1 | 94.9 | 95.5 | 95.9 |
| | 0.54 | 6.0 | 20.7 | 39.8 | 53.4 | 69.8 | 87.3 | 94.9 | 96.0 | 95.3 |
| | 1.10 | 4.0 | 26.1 | 30.7 | 50.1 | 67.4 | 86.6 | 94.5 | 95.8 | 96.4 |
| | 2.20 | 14.2 | 25.4 | 36.8 | 53.3 | 75.1 | 88.8 | 96.3 | 95.3 | 96.0 |
| | 4.30 | 22.2 | 29.8 | 43.5 | 53.6 | 75.5 | 88.1 | 96.3 | 95.7 | 96.5 |
| | 8.60 | 33.4 | 42.9 | 51.3 | 57.1 | 78.8 | 88.6 | 96.8 | 97.4 | 97.3 |
| | 17.00 | 46.2 | 51.3 | 51.2 | 63.0 | 79.2 | 88.2 | 97.4 | 97.0 | 97.3 |
| | 35.00 | 45.5 | 59.9 | 56.2 | 68.7 | 81.2 | 91.8 | 97.4 | 98.0 | 97.6 |

### EXAMPLE 20

### The Combination of Cyclosporine A and Ethopropazine Reduces IL-2 Secretion In Vitro

IL-2 secretion was measured by ELISA as described above after stimulation with phorbol 12-myristate 13-acetate and ionomycin. The effects of varying concentrations of cyclosporine A, ethopropazine, and a combination of ethopropazine and cyclosporine A were compared to control wells. These wells were stimulated with phorbol 12-myristate 13-acetate and ionomycin, but did not receive cyclosporine A or ethopropazine.

The results of this experiment are shown in Table 23. The effects of the agents alone and in combination are shown as percent inhibition of IL-2 secretion. These results are the average of experiments carried out with white blood cells obtained from two donors.

TABLE 23
% Inhibition

| | | Cyclosporine A (μM) | | | | | | | | |
|---|---|---|---|---|---|---|---|---|---|---|
| | | 0.00 | 0.01 | 0.02 | 0.03 | 0.06 | 0.12 | 0.25 | 0.50 | 0.99 |
| Ethopropazine (μM) | 0.00 | −12.7 | −2.5 | −3.7 | 22.1 | 29.3 | 75.7 | 91.0 | 92.1 | 92.2 |
| | 0.27 | −8.0 | −1.4 | 2.3 | 26.1 | 32.6 | 73.9 | 87.7 | 92.0 | 89.3 |
| | 0.54 | −0.7 | 3.2 | 3.3 | 26.4 | 43.9 | 76.5 | 88.3 | 92.5 | 92.8 |
| | 1.10 | −9.5 | 19.1 | 8.1 | 25.5 | 43.8 | 77.3 | 89.6 | 93.8 | 93.8 |
| | 2.20 | −10.4 | 16.1 | 8.7 | 24.8 | 56.0 | 79.7 | 91.3 | 93.9 | 94.0 |
| | 4.30 | −6.3 | 15.6 | 10.3 | 28.8 | 64.7 | 89.8 | 91.3 | 93.6 | 94.5 |
| | 8.60 | 19.5 | 15.0 | 32.2 | 48.3 | 81.8 | 92.1 | 94.2 | 95.3 | 95.3 |
| | 17.00 | 21.0 | 23.6 | 53.8 | 68.3 | 90.4 | 95.7 | 96.3 | 96.6 | 96.1 |
| | 35.00 | 52.3 | 80.5 | 89.2 | 92.9 | 96.9 | 97.3 | 97.0 | 97.5 | 98.1 |

### EXAMPLE 21

### The Combination of Cyclosporine A and Ethopropazine Reduces TNFα Secretion In Vitro

TNFα secretion was measured by ELISA as described above after stimulation with lipopolysaccharide. The effect of varying concentrations of cyclosporine A, ethopropazine, and cyclosporine A in combination with ethopropazine was compared to control wells stimulated without cyclosporine A or ethopropazine. The results of this experiment are shown in Table 24 below. The effects of the agents alone and in combination are shown as percent inhibition of TNFα secretion. These results are the average of experiments carried out with white blood cells obtained from two donors.

TABLE 24
% Inhibition

| | | Cyclosporine A (μM) | | | | | | | | |
|---|---|---|---|---|---|---|---|---|---|---|
| | | 0.00 | 0.01 | 0.02 | 0.03 | 0.06 | 0.12 | 0.25 | 0.50 | 0.99 |
| Ethopropazine (μM) | 0.00 | −14.1 | −10.6 | 2.2 | 39.1 | 71.6 | 89.4 | 95.2 | 96.2 | 96.1 |
| | 0.27 | −10.9 | 1.4 | 12.3 | 41.8 | 73.7 | 91.4 | 93.4 | 95.7 | 96.9 |
| | 0.54 | −13.9 | 1.5 | 8.7 | 42.6 | 74.7 | 89.8 | 94.7 | 96.7 | 96.3 |
| | 1.10 | −14.0 | −9.0 | 16.8 | 36.1 | 73.0 | 88.8 | 95.8 | 97.1 | 96.6 |
| | 2.20 | −5.5 | 9.5 | 23.4 | 52.6 | 81.3 | 92.1 | 95.8 | 96.1 | 96.5 |
| | 4.30 | −5.6 | 4.7 | 22.6 | 52.3 | 84.2 | 94.2 | 94.9 | 94.1 | 96.6 |
| | 8.60 | 12.0 | 24.8 | 61.3 | 72.7 | 89.9 | 94.3 | 94.9 | 93.2 | 93.9 |
| | 17.00 | 24.3 | 50.9 | 73.9 | 83.7 | 92.9 | 94.2 | 91.9 | 93.7 | 94.7 |
| | 35.00 | 69.6 | 88.7 | 93.8 | 95.8 | 97.5 | 97.0 | 96.7 | 96.1 | 97.4 |

### EXAMPLE 22

### The Combination of Cyclosporine A and Loperamide Reduces IL-2 Secretion In Vitro

IL-2 secretion was measured by ELISA as described above after stimulation with phorbol 12-myristate 13-acetate and ionomycin. The effects of varying concentrations of cyclosporine A, loperamide, and a combination of loperamide and cyclosporine A were compared to control wells. These wells were stimulated with phorbol 12-myristate 13-acetate and ionomycin, but did not receive cyclosporine A or loperamide.

The results of this experiment are shown in Table 25. The effects of the agents alone and in combination are shown as percent inhibition of IL-2 secretion. The results of this experiment are the average of experiments carried out with white blood cells obtained from two donors.

TABLE 25
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Loperamide (μM) | 0 | −13.0 | −0.8 | −3.2 | 10.5 | 36.8 | 76.1 | 91.9 | 92.9 | 93.9 |
| | 0.27 | −15.4 | −7.4 | −9.2 | 12.0 | 42.7 | 83.6 | 91.2 | 94.4 | 94.7 |
| | 0.54 | −15.4 | −10.3 | −7.8 | 6.1 | 49.8 | 82.1 | 92.0 | 94.2 | 92.2 |
| | 1.1 | −13.5 | −10.8 | −8.2 | 14.1 | 44.2 | 82.9 | 90.8 | 94.6 | 95.6 |
| | 2.2 | −14.9 | −12.2 | −3.1 | 28.4 | 59.7 | 83.7 | 90.1 | 91.8 | 94.6 |
| | 4.3 | −15.5 | −12.4 | 5.4 | 29.0 | 66.6 | 86.0 | 92.1 | 93.8 | 94.9 |
| | 8.6 | −10.5 | −5.1 | 6.8 | 42.7 | 79.8 | 91.7 | 94.2 | 95.5 | 96.1 |
| | 17 | 4.2 | 17.6 | 28.0 | 72.4 | 91.5 | 94.9 | 95.9 | 96.2 | 96.3 |
| | 35 | 42.4 | 67.0 | 83.3 | 92.1 | 96.9 | 96.9 | 97.3 | 97.4 | 96.6 |

### EXAMPLE 23

### The Combination of Cyclosporine A and Loperamide Reduces TNFα Secretion In Vitro

TNFα secretion was measured by ELISA as described above after stimulation with lipopolysaccharide. The effect of varying concentrations of cyclosporine A, loperamide, and cyclosporine A in combination with loperamide was compared to control wells stimulated without cyclosporine A or loperamide. The results of this experiment are shown in Table 26. The effects of the agents alone and in combination are shown as percent inhibition of TNFα secretion. The results of this experiment are the average of experiments carried out with white blood cells obtained from two donors.

TABLE 26
% Inhibition

| | | Cyclosporine A (μM) | | | | | | | | |
|---|---|---|---|---|---|---|---|---|---|---|
| | | 0 | 0.0077 | 0.015 | 0.031 | 0.062 | 0.12 | 0.25 | 0.5 | 0.99 |
| Loperamide (μM) | 0 | −5.5 | 14.8 | 34.2 | 57.1 | 80.5 | 91.8 | 96.3 | 96.3 | 96.3 |
| | 0.27 | 2.5 | 17.9 | 35.8 | 54.9 | 79.5 | 92.0 | 95.9 | 96.5 | 96.5 |
| | 0.54 | −5.7 | 24.6 | 42.7 | 49.5 | 85.2 | 93.4 | 96.1 | 96.4 | 96.0 |
| | 1.1 | −1.0 | 26.6 | 41.0 | 54.8 | 79.4 | 92.8 | 95.3 | 95.5 | 95.8 |
| | 2.2 | −6.5 | 27.4 | 37.8 | 71.9 | 83.8 | 94.9 | 95.8 | 95.6 | 97.0 |
| | 4.3 | 7.6 | 27.6 | 43.4 | 76.1 | 91.1 | 95.6 | 96.6 | 96.2 | 96.5 |
| | 8.6 | 22.0 | 43.3 | 65.8 | 78.3 | 94.7 | 96.7 | 97.2 | 97.3 | 97.4 |
| | 17 | 56.2 | 73.1 | 84.7 | 92.4 | 97.1 | 97.6 | 97.6 | 98.2 | 98.2 |
| | 35 | 87.3 | 94.2 | 96.3 | 97.7 | 98.8 | 98.8 | 98.9 | 99.0 | 98.7 |

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

What is claimed is:

1. A composition comprising a non-steroidal immunophilin-dependent immunosuppressant (NsIDI) and an NsIDI enhancer (NsIDIE) in amounts that together are sufficient in vivo to decrease proinflammatory cytokine secretion or production or to treat an immunoinflammatory disorder.

2. The composition of claim 1, wherein said NsIDI is a calcineurin inhibitor.

3. The composition of claim 2, wherein said calcineurin inhibitor is cyclosporine, tacrolimus, ascomycin, pimecrolimus, or ISAtx247.

4. The composition of claim 1, wherein said said NsIDI is an FK506-binding protein.

5. The composition of claim 4, wherein said FK506-binding protein is rapamycin or everolimus.

6. The composition of claim 1, wherein said NsIDIE is a selective serotonin reuptake inhibitor (SSR₁), a tricyclic antidepressant (TCA), a phenoxy phenol, an antihistamine, a phenothiazine, or a mu opioid receptor agonist.

7. The composition of claim 6, wherein said SSRI is selected from the group consisting of fluoxetine, sertraline, paroxetine, fluvoxamine, citalopram, and escitalopram.

8. The composition of claim 6, wherein said TCA is selected from the group consisting of maprotiline, nortriptyline, protriptyline, desipramine, amitriptyline, amoxapine, clomipramine, dothiepin, doxepin, desipramine, imipramine, lofepramine, mianserin, oxaprotiline, octriptyline, and trimipramine.

9. The composition of claim 6, wherein said phenoxy phenol is triclosan.

10. The composition of claim 6, wherein said antihistamine is selected from the group consisting of ethanolamines, ethylenediamines, phenothiazines, alkylamines, piperazines, piperidines, and atypical antihistamines.

11. The composition of claim 6, wherein said antihistamine is selected from the group consisting of desloratadine, thiethylperazine, bromodiphenhydramine, promethazine, cyproheptadine, loratadine, clemizole, azatadine, cetirizine, chlorpheniramine, dimenhydramine, diphenydramine, doxylamine, fexofenadine, meclizine, pyrilamine, and tripelennamine.

12. The composition of claim 6, wherein said phenothiazine is chlorpromazine or ethopropazine.

13. The composition of claim 6, wherein said mu opioid receptor agonist is a piperidine butyramide derivative.

14. The composition of claim 6, wherein said mu opioid receptor agonist is loperamide, meperidine, or diphenoxylate.

15. The composition of claim 1, wherein said composition further comprises a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, non-steroidal calcineurin inhibitor, vitamin D analog, psoralen, retinoid, or 5-amino salicylic acid.

16. The composition of claim 15, wherein said NSAID is ibuprofen, diclofenac, or naproxen.

17. The composition of claim 15, wherein said COX-2 inhibitor is rofecoxib, celecoxib, valdecoxib, or lumiracoxib.

18. The composition of claim 15, wherein said biologic is adelimumab, etanercept, or infliximab.

19. The composition of claim 15, wherein said DMARD is methotrexate or leflunomide.

20. The composition of claim 15, wherein said xanthine is theophylline.

21. The composition of claim 15, wherein said anticholinergic compound is ipratropium or tiotropium.

22. The composition of claim 15, wherein said beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline.

23. The composition of claim 15, wherein said vitamin D analog is calcipotriene or calcipotriol.

24. The composition of claim 15, wherein said psoralen is methoxsalen.

25. The composition of claim 15, wherein said retinoid is acitretin or tazoretene.

26. The composition of claim 15, wherein said 5-amino salicylic acid is mesalamine, sulfasalazine, balsalazide disodium, or olsalazine sodium.

27. The composition of claim 15, wherein said small molecule immunomodulator is VX 702, SCIO 469, doramapimod, RO 30201195, SCIO 323, DPC 333, pranalcasan, mycophenolate, or merimepodib.

28. The composition of claim 1, wherein said composition is formulated for topical administration.

29. The composition of claim 1, wherein said composition is formulated for systemic administration.

30. A method of decreasing proinflammatory cytokine secretion or production in a patient, said method comprising administering to the patient an NsIDI and an NsIDIE simultaneously or within 14 days of each other in amounts sufficient in vivo to decrease proinflammatory cytokine secretion or production in said patient.

31. A method for treating a patient diagnosed with or at risk of developing an immunoinflammatory disorder, said method comprising administering to the patient an NsIDI and an NsIDIE simultaneously or within 14 days of each other in amounts sufficient to treat said patient.

32. The method of claim 31, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, or psoriatic arthritis.

33. The method of claim 31, wherein said NsIDI is cyclosporine, tacrolimus, ISAtx247, ascomycin, pimecrolimus, rapamycin, or everolimus.

34. The method of claim 31, where said NsIDIE is an SSR₁, a TCA, a phenoxy phenol, an antihistamine, a phenothiazine, or a mu opioid receptor agonist.

35. The method of claim 34, wherein said SSRI is selected from the group consisting of fluoxetine, sertraline, paroxetine, fluvoxamine, citalopram, and escitalopram.

36. The method of claim 34, wherein said TCA is selected from the group consisting of maprotiline, nortriptyline, protriptyline, desipramine, amitriptyline, amoxapine, clomipramine, dothiepin, doxepin, desipramine, imipramine, lofepramine, mianserin, oxaprotiline, octriptyline, and trimipramine.

37. The method of claim 34, wherein said phenoxy phenol is triclosan.

38. The method of claim 34, wherein said antihistamine is selected from the group consisting of desloratadine, thiethylperazine, bromodiphenhydramine, promethazine, cyproheptadine, loratadine, clemizole, azatadine, cetirizine, chlorpheniramine, dimenhydramine, diphenydramine, doxylamine, fexofenadine, meclizine, pyrilamine, and tripelennamine.

39. The method of claim 34, wherein said phenothiazine is chlorpromazine or ethopropazine.

40. The method of claim 34, wherein said mu opioid receptor agonist is loperamide, meperidine, or diphenoxylate.

41. The method of claim 31, wherein said method further comprises administering an NSAID, COX-2 inhibitor, biologic, DMARD, xanthine, anticholinergic compound, beta receptor agonist, bronchodilator, non-steroidal calcineurin inhibitor, vitamin D analog, psoralen, retinoid, or 5-amino salicylic acid.

42. The method of claim 41, wherein said NSAID is ibuprofen, diclofenac, or naproxen.

43. The method of claim 41, wherein said COX-2 inhibitor is rofecoxib, celecoxib, valdecoxib, or lumiracoxib.

44. The method of claim 41, wherein said biologic is adelimumab, etanercept, or infliximab.

45. The method of claim 41, wherein said DMARD is methotrexate or leflunomide.

46. The method of claim 41, wherein said xanthine is theophylline.

47. The method of claim 41, wherein said anticholinergic compound is ipratropium or tiotropium.

48. The method of claim 41, wherein said beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline.

49. The method of claim 41, wherein said vitamin D analog is calcipotriene or calcipotriol.

50. The method of claim 41, wherein said psoralen is methoxsalen.

51. The method of claim 41, wherein said retinoid is acitretin or tazoretene.

52. The method of claim 41, wherein said 5-amino salicylic acid is mesalamine, sulfasalazine, balsalazide disodium, or olsalazine sodium.

53. The method of claim 31, wherein said composition is formulated for topical administration.

54. The method of claim 31, wherein said composition is formulated for systemic administration.

55. A method of decreasing proinflammatory cytokine secretion or production in a cell, said method comprising contacting said cell with an NsIDI and an NsIDIE simultaneously or within 14 days of each other in amounts sufficient in vivo to decrease proinflammatory cytokine secretion or production in said cell.

56. The method of claim 55, wherein said cell is a mammalian cell in vivo.

57. A kit, comprising:
(i) a composition comprising an NsIDI and an NsIDIE; and
(ii) instructions for administering said composition to a patient diagnosed with or at risk of developing an immunoinflammatory disorder.

58. A kit, comprising:
(i) an NsIDI;
(ii) an NsIDIE; and
(iii) instructions for administering said NsIDI and said NsIDIE to a patient diagnosed with or at risk of developing an immunoinflammatory disorder.

59. A kit comprising:
(i) an NsIDI; and
(ii) instructions for administering said NsIDI and an NsIDIE to a patient diagnosed with or at risk of developing an immunoinflammatory disorder.

60. A kit comprising:
(i) an NsIDIE; and
(ii) instructions for administering said NsIDIE and an NsIDI to a patient diagnosed with or at risk of developing an immunoinflammatory disorder.

61. A method for identifying combinations of compounds useful for suppressing the secretion of proinflammatory cytokines in a patient in need of such treatment, said method comprising the steps of:
(a) contacting cells in vitro with an NsIDI and a candidate compound; and
(b) determining whether the combination of said NsIDI and said candidate compound reduces cytokine levels in blood cells stimulated to secrete the cytokines relative to cells contacted with said NsIDI but not contacted with said candidate compound or cells contacted with said candidate compound but not with said NsIDI, wherein a reduction of said cytokine levels identifies said combination as a combination that is useful for treating a patient in need of such treatment.
